(19) United States
(12) Patent Application Publication
(10) Pub. No.: US 2025/0205107 A1
(43) Pub. Date: Jun. 26, 2025

(54) PNEUMATIC COMPRESSION VEST DEVICE, METHOD, AND SYSTEM FOR TRANSTHORACIC MANIPULATION FOR OXYGENATION

(71) Applicants: GEORGIA TECH RESEARCH CORPORATION, Atlanta, GA (US); EMORY UNIVERSITY, Atlanta, GA (US)

(72) Inventors: Frank L. HAMMOND, III, Atlanta, GA (US); Alexander B. AMBROSE, Atlanta, GA (US); Harold SOLOMON, Atlanta, GA (US); Maxwell WEINMANN, Atlanta, GA (US)

(21) Appl. No.: 18/847,528

(22) PCT Filed: Mar. 16, 2023
(86) PCT No.: PCT/US2023/015376

§ 371 Date: Sep. 16, 2024

Related U.S. Application Data
(60) Provisional application No. 63/320,485, filed on Mar. 16, 2022.

(51) Int. Cl. A61H 9/00

(52) U.S. Cl. CPC A61H 9/0007; A61H 9/0078; A61H 2201/0157; A61H 2201/10; A61H 2201/1621; A61H 2201/1626; A61H 2201/5007; A61H 2201/5023; A61H 2201/5071; A61H 2201/5089; A61H 2230/201; A61H 2230/305; A61H 2230/405

(57) ABSTRACT

An exemplary method and pneumatic vest system (also referred to herein as a V/Q vest) are disclosed for improving oxygenation that can reproduce the effects of proning treatment. The exemplary pneumatic vest can be used synergistically with proning, as a replacement therapy for proning treatment, and/or to monitor or assess whether patients would respond well to a proning treatment. The exemplary pneumatic vest can be integrated with sensors and intubation equipment. The compression vest system can integrate a mechanical ventilator with a pneumatic compression vest to improve oxygenation, provide potential alternative or bridge to proning patients, and/or reduce mechanical ventilatory morbidity.

### RELATED APPLICATION

This PCT application claims priority to, and the benefit of, U.S. Provisional Patent Application No. 63/320,485, filed Mar. 16, 2022, entitled “V/Q VEST ASSISTED VENTILATION,” which is hereby incorporated by reference herein in its entirety.

### FIELD OF THE DISCLOSURE

The present disclosure generally relates to the oxygenation of a patient and, more particularly, to a vest, a ventilator, and interconnections and complimentary interactions between the vest and the ventilator in such a way as to improve oxygenation ratios for the patient.

### BACKGROUND

Critical care patients who experience acute respiratory distress syndrome, or other respiratory diseases or conditions, are commonly placed on mechanical ventilators to improve oxygen delivery and overall gas exchange of the pulmonary system. With the pulmonary inflammation typically accompanying acute respiratory distress syndrome (ARDS), patients can experience disruption in lung function culminating in profound reductions in blood oxygen levels. This is most commonly due to abnormalities of ventilation and perfusion within the lung due to the initiating pathology, be it toxic, infectious, traumatic etc. When unresponsive to traditional mechanical ventilator support, the patient is placed upon their abdomen; a process known as proning. Among other things, proning produces a greater homogeneity within the lung facilitating matching between perfusion and ventilation resulting in improved oxygenation. In these cases, the prone position can facilitate improved blood flow to portions of the lung that were not previously participating in the gas exchange process.

Proning is the process of turning a patient with precise, safe motions from their back onto their abdomen (stomach) so the individual is lying face down. While beneficial, proning a patient can carry significant risks: for example, airway obstruction, dislodgement of endotracheal tube, pressure-related skin injuries, facial and airway edema (swelling), hypotension (low blood pressure), and arrhythmias (irregular heartbeat/rate). The process additionally requires several hospital staff members, e.g., 6-7 staff members, to carry out and does not guarantee an improvement in the patient's condition.

A need, therefore, exists for safer and improved systems and methods for oxygenating a patient, which may overcome one or more of the above-mentioned challenges associated with existing techniques.

### SUMMARY

An exemplary method and pneumatic vest system (also referred to herein as a V/Q vest) are disclosed for improving oxygenation that can reproduce some, and potentially all of the effects of proning treatment. The exemplary pneumatic vest can be used synergistically with proning, as a replacement therapy for proning treatment, and/or to monitor or assess whether patients would respond well to a proning treatment. The exemplary pneumatic vest can be integrated with sensors and intubation equipment including, but not limited to, electronic sensors (e.g., electrical impedance sensors, near Infrared sensors, oximetric sensors, sonographic sensors, pressure sensors, and/or the like). Such sensors can be configured to evaluate lung pressures, sounds, fluid content, and pressure generated by the pneumatic vest system. The compression vest system can integrate a mechanical ventilator with a pneumatic compression vest to improve oxygenation, provide a potential alternative or bridge to proning patients, and/or reduce mechanical ventilatory morbidity.

A study was conducted to evaluate the proning treatment using pneumatic vests. In the study, a manual vest was initially employed. An electronically controlled pneumatic vest has been developed and employed. The manual vest may be employed to assess whether patients would respond well to a proning treatment.

In an aspect, the method can comprise: receiving, by a controller operatively coupled to a wearable pneumatic vest, a control input (e.g., from a local sensor, another device in communication with the controller) indicative of a detected oxygen level in the patient's body or a user-selected target oxygen level (e.g., received via manual input or by adjusting pneumatic vest settings); determining, by the controller, a plurality of operating parameters for the wearable pneumatic vest based at least in part on the detected oxygen level or the user-selected target oxygen level; and regulating, by the controller, an amount of pressure applied to at least a portion of the patient's anterior, posterior, and lateral chest walls by the wearable pneumatic vest based at least in part on the plurality of operating parameters.

In some embodiments, the method further comprises receiving, by the controller, oxygenation data obtained via at least one other device, wherein the plurality of operating parameters are determined based at least in part on the oxygenation data.

In some implementations, the wearable pneumatic vest comprises a plurality of inflatable bladders.

In some embodiments, the plurality of operating parameters comprises a duty cycle for each of a plurality of inflatable bladders.

In some embodiments, determining the plurality of operating parameters comprises: identifying, by the controller and based at least in part on the oxygenation data, the detected oxygen level, or the user-selected target oxygen level, a target location of the patient's anterior, posterior, and lateral chest walls, and a corresponding pressure value; selecting, by the controller, at least one of the plurality of inflatable bladders associated with the target location; and causing, by the controller, at least one of the plurality of inflatable bladders to inflate or deflate to reach the corresponding pressure value.

In some embodiments, the method further comprises: receiving, by the controller, sensor data for the patient; adjusting, by the controller, the plurality of operating parameters based at least in part on the sensor data; and modifying, by the controller, the amount of pressure applied to at least a portion of the patient's anterior, posterior, and lateral chest walls by the wearable vest based at least in part on the adjusted operating parameters.

In some embodiments, the sensor data comprises at least one of an internal pressure value for each of a plurality of inflatable bladders, vibration data, electrical impedance data, oximetric data, Near infrared spectroscopy (NIRS) data, audio data, or an applied amount of pressure to at least a portion of the patient's anterior, posterior, and lateral chest walls.

In accordance with certain embodiments, a wearable pneumatic vest is provided. The wearable pneumatic vest can include: a device body comprising a plurality of independently controllable inflatable bladders; a restriction element configured to secure the device body to at least a portion of the patient's anterior, posterior, and lateral chest walls, and minimize pressure variations throughout the patient's breathing cycle; and a controller operatively coupled to the plurality of inflatable bladders, the controller being configured to regulate an amount of pressure applied to at least a portion of the patient's anterior, posterior, and lateral chest walls by controlling inflation and deflation of each of the plurality of inflatable bladders.

In some embodiments, the controller is configured to control the inflation and deflation of each of the plurality of inflatable bladders based at least in part on oxygenation data indicative of a detected oxygen level in the patient's body or a user-selected target oxygen level.

In some embodiments, the restriction element comprises one or more removable straps attached to an anterior surface of the device body.

In some embodiments, the wearable pneumatic vest further comprises at least one sensor array located on each internal surface of the wearable pneumatic vest.

In some embodiments, the at least one sensor array is configured to monitor at least one of the localized transcutaneous pressure values, cardiovascular pressure values, and an esophageal pressure value.

In some embodiments, the at least one sensor array comprises at least one of a pressure sensor, a vibration sensor, and an audio sensor.

In some embodiments, each of the plurality of inflatable bladders is operatively coupled to a respective pressure sensor via a pneumatic connector, and wherein each respective pressure sensor is configured to monitor an internal pressure value for one of the plurality of inflatable bladders.

In some embodiments, the controller is further configured to control the inflation and deflation of each of the plurality of inflatable bladders based at least in part on sensor data obtained from the at least one sensor array.

In some embodiments, the controller comprises a Proportional-Integral-Derivative (PID) controller that is configured to drive an internal pressure of each of the plurality of inflatable bladders to a specified internal pressure based at least in part on the oxygenation data or the user-selected target oxygen level.

In some embodiments, the wearable pneumatic vest further comprises a graphical user interface in electronic communication with the controller that is configured to receive user inputs and facilitate monitoring of the patient.

In accordance with certain embodiments, a system for optimizing patient oxygenation is provided. The system can comprise: a pneumatic vest configured for applying pressure to at least a portion of the patient's anterior, posterior, and lateral chest walls; and a controller in operable communication with the ventilator and the pneumatic vest, the controller configured to regulate the pressure applied by the pneumatic vest to the patient's anterior, posterior and lateral chest walls.

In some embodiments, the system includes a ventilator configured for delivering varying concentrations of oxygen to the patient's lungs, the controller having an interface to communicate with the ventilator.

In some embodiments, the controller is in operable communication with at least one of a pulse oximeter, a blood oxygen monitor, and ventilation equipment to facilitate measurement of at least one of lung mechanics, impact on cardiovascular function, and intra-abdominal pressure.

In some embodiments, the controller is configured to regulate the pressure applied by the pneumatic vest based at least in part on one or more ventilator operating parameters (e.g., a ventilator mode, a pressure, a rate, a tidal volume, a peak flow, a positive end-expiratory pressure, a fractional concentration of oxygen, and an inspiratory time).

The present disclosure provides systems and methods for oxygenating a patient. In one aspect, a system for oxygenating a patient is provided. In one embodiment, the system may include a ventilator configured for delivering varying concentrations of oxygen to the patient's lungs, a wearable pneumatic vest configured for applying pressure to the patient's body (e.g., anterior, posterior, and lateral chest walls), and a controller in operable communication with the ventilator and the wearable pneumatic vest.

The controller may be configured to regulate the pressure applied by the wearable pneumatic vest to the patient's body (e.g., anterior, posterior, and lateral chest walls).

In some embodiments, the controller may be further configured to receive oxygenation data indicative of a detected oxygen level in the patient's body and regulate the pressure applied by the wearable pneumatic vest to the patient's body (e.g., anterior, posterior, and lateral chest walls) based at least in part on the detected oxygen level.

In some embodiments, the controller may be further configured to receive a target oxygen level for the patient and regulate the pressure applied by the wearable pneumatic vest to the patient's body (e.g., anterior, posterior, and lateral chest walls) based at least in part on the detected oxygen level and the target oxygen level.

In some embodiments, the controller may be further configured to determine that the detected oxygen level is greater than the target oxygen level and cause the pressure applied by the wearable pneumatic vest to the patient's body (e.g., anterior, posterior, and lateral chest walls) to be decreased based at least in part on the determination that the detected oxygen level is greater than the target oxygen level.

In some embodiments, the controller may be further configured to determine that the detected oxygen level is less than the target oxygen level and cause the pressure applied by the wearable pneumatic vest to the patient's body (e.g., anterior, posterior, and lateral chest walls) to be increased based at least in part on the determination that the detected oxygen level is less than the target oxygen level.

In some embodiments, the system also may include a blood oxygen monitor configured for detecting the oxygen level of the patient's blood. The controller may be in operable communication with the blood oxygen monitor, and the controller may be further configured to receive the oxygenation data from the blood oxygen monitor.

In some embodiments, the system also may include a user interface configured to allow a clinician to input the oxygenation data.

The controller may be in operable communication with the user interface, and the controller may be further configured to receive the oxygenation data from the user interface. In some embodiments, the system also may include a cardiovascular pressure monitor configured for detecting the cardiovascular pressure of the patient.

The controller may be in operable communication with the cardiovascular pressure monitor, and the controller may be further configured to regulate the pressure applied by the wearable pneumatic vest to the patient's body (e.g., anterior, posterior, and lateral chest walls) based at least in part on the cardiovascular pressure. In some embodiments, the system also may include an esophageal pressure monitor configured for detecting the esophageal pressure of the patient.

The controller may be in operable communication with the esophageal pressure monitor, and the controller may be further configured to regulate the pressure applied by the wearable pneumatic vest to the patient's body (e.g., anterior, posterior, and lateral chest walls) based at least in part on the esophageal pressure.

In some embodiments, the system also may include one or more pressure sensors disposed on an undersurface of the wearable pneumatic vest and configured for detecting a localized transcutaneous pressure applied to the patient.

The controller may be in operable communication with the one or more pressure sensors, and the controller may be further configured to regulate the pressure applied by the wearable pneumatic vest to the patient's body (e.g., anterior, posterior, and lateral chest walls) based at least in part on the localized transcutaneous pressure.

In some embodiments, the controller may be further configured to receive ventilator data indicative of one or more operating parameters of the ventilator and regulate the pressure applied by the wearable pneumatic vest to the patient's body (e.g., anterior, posterior, and lateral chest walls) based at least in part on the one or more operating parameters of the ventilator.

In some embodiments, the controller may be further configured to receive the ventilator data from the ventilator.

In some embodiments, the system also may include a user interface configured to allow a clinician to input the ventilator data.

The controller may be in operable communication with the user interface, and the controller may be further configured to receive the ventilator data from the user interface.

In some embodiments, the one or more operating parameters of the ventilator may include one or more of a ventilator mode, a pressure, a rate, a tidal volume, a peak flow, a positive end-expiratory pressure, a fractional concentration of oxygen, and an inspiratory time.

In some embodiments, the controller may be further configured to determine a respiratory cycle of the patient corresponding to the oxygen delivered by the ventilator to the patient's lungs and regulate the pressure applied by the wearable pneumatic vest to the patient's body (e.g., anterior, posterior, and lateral chest walls) based at least in part on the respiratory cycle.

In some embodiments, the controller may be further configured to decrease the pressure applied by the wearable pneumatic vest to the patient's body (e.g., anterior, posterior, and lateral chest walls) at the beginning of the respiratory cycle.

In some embodiments, the controller may be further configured to increase the pressure applied by the wearable pneumatic vest to the patient's body (e.g., anterior, posterior, and lateral chest walls) at the end of the respiratory cycle.

In some embodiments, the wearable pneumatic vest may include one or more chambers configured for being inflated and deflated to apply pressure to the patient's body (e.g., anterior, posterior, and lateral chest walls).

In some embodiments, the controller may be further configured to regulate the pressure applied by the wearable pneumatic vest to the patient's body (e.g., anterior, posterior, and lateral chest walls) by regulating an inflation pressure of the wearable pneumatic vest.

In some embodiments, the controller may be further configured to receive one or more inflation pressure settings and regulate the inflation pressure of the wearable pneumatic vest based at least in part on the one or more inflation pressure settings.

In some embodiments, the one or more inflation pressure settings may include a maximum inflation pressure setting corresponding to a maximum inflation pressure for the wearable pneumatic vest, and the controller may be further configured to determine that the inflation pressure of the wearable pneumatic vest is greater than the maximum inflation pressure and cause the inflation pressure of the wearable pneumatic vest to be decreased below the maximum inflation pressure.

In some embodiments, the controller may be further configured to cause an alarm to be activated based at least in part on the determination that the inflation pressure of the wearable pneumatic vest is greater than the maximum inflation pressure.

In some embodiments, the one or more inflation pressure settings may include a minimum inflation pressure setting corresponding to a minimum inflation pressure for the wearable pneumatic vest, and the controller may be further configured to determine that the inflation pressure of the wearable pneumatic vest is less than the minimum inflation pressure, and cause the inflation pressure of the wearable pneumatic vest to be increased above the minimum inflation pressure.

In some embodiments, the controller may be further configured to cause an alarm to be activated based at least in part on the determination that the inflation pressure of the wearable pneumatic vest is less than the minimum inflation pressure.

In some embodiments, the controller may be further configured to incrementally regulate the inflation pressure of the wearable pneumatic vest based at least in part on the one or more inflation pressure settings.

In some embodiments, the system also may include a user interface configured for allowing a clinician to input the one or more inflation pressure settings, the controller may be in operable communication with the user interface, and the controller may be further configured to receive the one or more inflation pressure settings from the user interface.

In some embodiments, the system also may include a pump in fluid communication with the one or more chambers. The controller may be in operable communication with the pump, and the controller may be further configured to control the pump to inflate and deflate the one or more chambers.

In some embodiments, the one or more chambers may include a plurality of chambers configured for being independently inflated and deflated to apply different pressures to different regions of the patient's body (e.g., anterior, posterior, and lateral chest walls).

In some embodiments, the controller may be further configured to determine one or more operating parameters of the ventilator, and adjust one or more operating parameters of the wearable pneumatic vest based at least in part on the one or more operating parameters of the ventilator.

In some embodiments, the controller may be further configured to determine one or more operating parameters of the wearable pneumatic vest, and adjust one or more operating parameters of the ventilator based at least in part on the one or more operating parameters of the wearable pneumatic vest.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure.

FIGS. 1A and 1B each is a schematic diagram depicting an exemplary pneumatic compression vest system (V/Q vest) for transthoracic manipulation for optimizing oxygenation for a patient in accordance with an exemplary embodiment.

FIGS. 2A, 2B, and 2C are schematic diagrams depicting an example pneumatic compression vest according to an illustrative embodiment.

FIGS. 3A, 3B, and 3C are schematic diagrams depicting a second example pneumatic compression vest according to an illustrative embodiment.

FIG. 4 shows a third example pneumatic compression vest according to an illustrative embodiment.

FIG. 5 is a schematic diagram showing a free-body diagram of a simplified vest of the V/Q vest according to an illustrative embodiment.

FIGS. 6A and 6B each shows an example configuration of the V/Q vest with sensor arrays according to an illustrative embodiment.

FIG. 7 is a flowchart diagram depicting an example method for improving oxygenation, being either a synergistic mode with proning or replacement therapy.

FIG. 8A shows a diagram of an example control architecture for the V/Q vest according to an illustrative embodiment.

FIG. 8B shows an example graphic user interface for an electro-pneumatic controller of the V/Q vest according to an illustrative embodiment.

FIGS. 9A, 9B, 9C, 9D, and 9E show experimental results for performance of two prototype V/Q vests employed in a study to optimize oxygenation for a patient according to an illustrative embodiment.

FIGS. 10A and 10B show experimental results for a third prototype V/Q vests in an additional study to optimize oxygenation for a patient according to an illustrative embodiment.

### DETAILED SPECIFICATION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well-known methods, procedures, components, and circuitry have not been described in detail so as not to unnecessarily obscure aspects of the embodiments.

The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### Example System

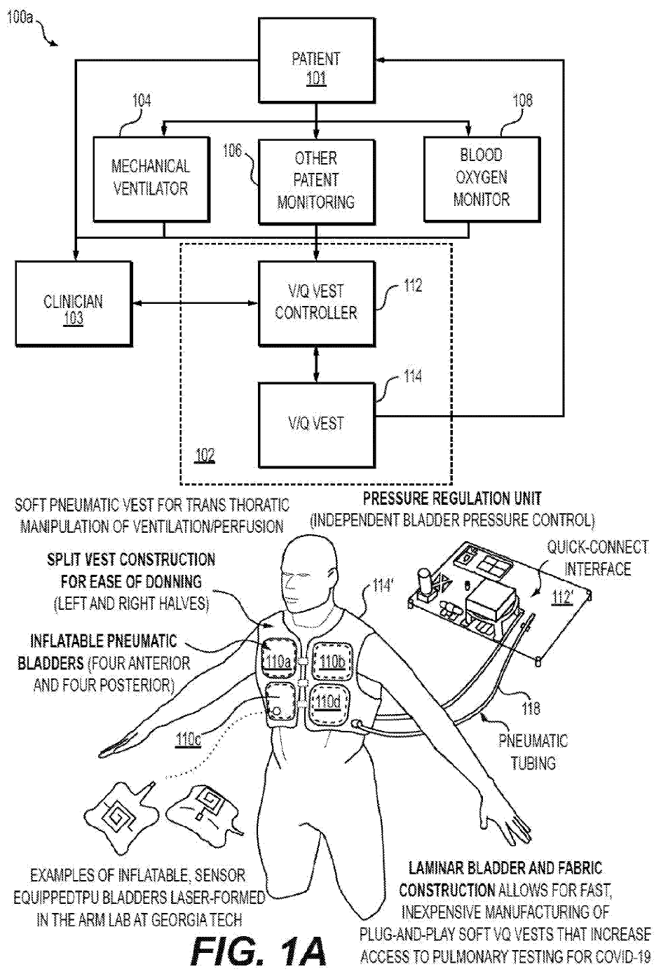

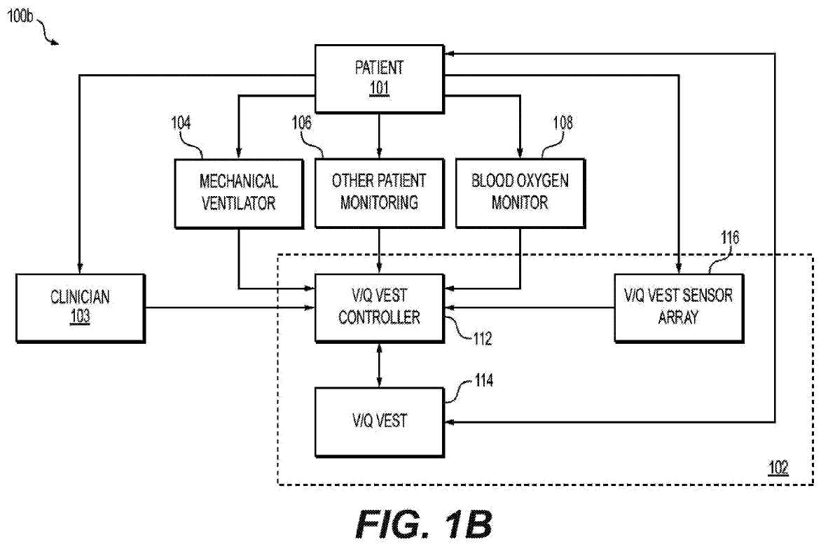

FIG. 1A is a schematic diagram depicting an exemplary pneumatic compression vest system 100 (shown as 100a) for transthoracic manipulation for optimizing oxygenation for a patient 101 in accordance with an exemplary embodiment. As shown in FIG. 1A, the system 100a includes a pneumatic compression vest device 102 that can operate with a mechanical ventilator 104, one or more patient monitoring devices 106 (e.g., a pulse oximeter), and a blood oxygen monitor 108.

The mechanical ventilator 104 is configured to deliver varying concentrations of oxygen to the patient's lungs. The patient monitoring device 106 is configured as a pulse oximeter to detect blood oxygen saturation. The blood oxygen monitor 108 is configured to detect the oxygen level of the patient's blood.

The pneumatic compression vest device 102, preferably, includes a controller 112 and a wearable pneumatic compression vest 114 comprising a plurality of inflatable bladders 110 (shown as 110a, 110b, 110c, 110d) to apply pressure to at least a portion of a patient's body (e.g., back, upper abdomen, thorax, and/or the like). The controller 112 is in operable communication with the wearable vest 114, e.g., via a quick-connect interface, configured to interface with each of the plurality of inflatable bladders via at least one pneumatic tubing 118. The controller 112 may be in operable communication with a cardiovascular pressure monitor, and the controller 112 may be further configured to regulate the pressure applied by the wearable pneumatic vest to the patient's body as per additional input from electrical impedance monitors placed on the internal surface of the wearable pneumatic compression vest 114.

The controller 112 is configured to receive a sensor input or a command from a user to executes a control loop (e.g., Proportional-Integral-Derivative (PID) control) configured to regulate pneumatic compression operations of the wearable vest 114 by controlling a pump (not shown) that regulates the amount of pressure applied by the wearable vest 114 to the patient's body (e.g., anterior, posterior, and lateral chest walls), e.g., based at least in part on one or more ventilator operating parameters obtained from the mechanical ventilator 104 or from a user-defined input.

In some embodiments, the wearable pneumatic compression vest 114 is configured for manual operation.

Examples of the ventilator operating parameters include but are not limited to a ventilator mode, a pressure, a rate, a tidal volume, a peak flow, a positive end-expiratory pressure, a fractional concentration of oxygen, and an inspiratory time.

In some embodiments, the mechanical ventilator 104 includes ventilation equipment to facilitate measurement of at least one of lung mechanics, impact on cardiovascular function, and intra-abdominal pressure with respect to the patient 101. The role of the mechanical ventilator is to assist the ailing lung by “recruiting” marginally effected alveoli to continue to participate in the gas exchange process, thereby counteracting the impact of V/Q mismatch or any other lung pathology leading to impaired oxygenation. The clinician may titrate components of rate, volume, pressure, flow, and other parameters according to the physiologic response of the lung as measured by the pressure imparted on the patient via the vest, volume characteristics, by the sensors for flow and pressure within the ventilator, and also by the patient's Arterial Blood Gas (ABG).

At times, traditional mechanical ventilator therapeutic maneuvers may be inadequate and/or may place the damaged lung at risk of mechanical trauma due to pressure, volume, or rate. The vest may act by introducing a new “synergistic” noninvasive approach that acts via a number of mechanisms, including prevention of overdistension of alveoli, thereby optimizing alveolar mechanics and reducing the mismatch between areas of inflation/ventilation and blood flow (V/Q mismatch). This V/Q realignment may allow a reduction in pressure delivered by the ventilator, thereby reducing mechanical ventilator morbidity.

In some embodiments, the vest may provide external pressure to the anterolateral and abdominal thorax of the patient and supplementary to traditional ventilator support. Vest pressures may be titrated to 02 levels as determined by ABG's (and/or pulse oximetry) and per the clinicians' goals. Inflation pressures may be preprogrammed by the clinician within a clinically acceptable range. Resultant ABG's and/or oximetric pulse data also may be entered into the mechanical ventilator's oxygenation/vest software. Once goals have been achieved, oxygenation monitoring may continue to rapidly identify any fluctuations in patient oxygenation levels. Should oxygen levels exceed goals, vest pressure can be reduced via the vest controller algorithm. Similarly, should oxygenation decline, the vest controller can increase vest pressure within limits set by the clinician.

In some embodiments, the vest controller may constantly monitor the ventilator data. In addition, the impact of inflation may be systemically measured by constant monitoring of cardiovascular pressures to assess any hemodynamic impact. The ventilator also may measure lung mechanics, which include peak inspiratory pressures, alveolar pressure, compliance, etc. An esophageal pressure monitor, typically standard-of-care in respiratory failure, also may be connected to the ventilator and may be used to determine the effect of applying pressure through the vest across the patient's chest. Additional pressure sensors on the undersurface of the vest may be used to determine localized transcutaneous pressures.

In some embodiments, the vest may be configured to operate in each of an intelligent mode and a manual mode. When operating in the intelligent mode, the vest may operate intelligently and independently within the parameters selected by the clinician. When operating in manual mode, the vest may be driven manually by the clinician, who regulates the optimal settings of both the ventilator and the vest. In the manual mode, the clinician may manually adjust all aspects of the ventilator, such as ventilator mode, pressure, rate, tidal volume, peak flow, PEEP, F₁O₂, and inspiratory time (as described below), in concert with vest ventilation pressure changes to optimize oxygenation. Alternately, the clinician may set variable vest inflation pressures and observe the physiological impact on the background of fixed ventilator settings or vice versa. In the intelligent mode, the clinician may determine the level of support provided by the ventilator, which then determines volume, flow, pressure, etc. based on the particular ventilator's algorithm and the patient's physiologic performance during spontaneous efforts at breathing.

### Example Pneumatic Compression Devices

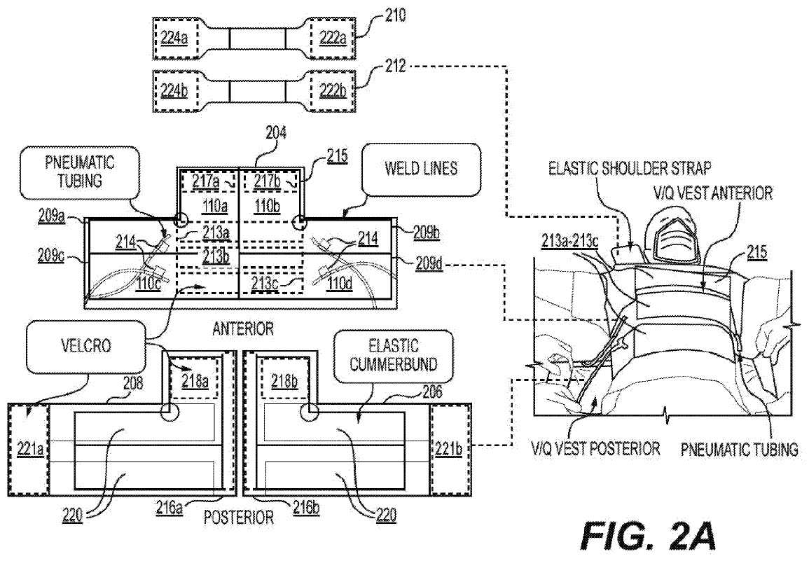

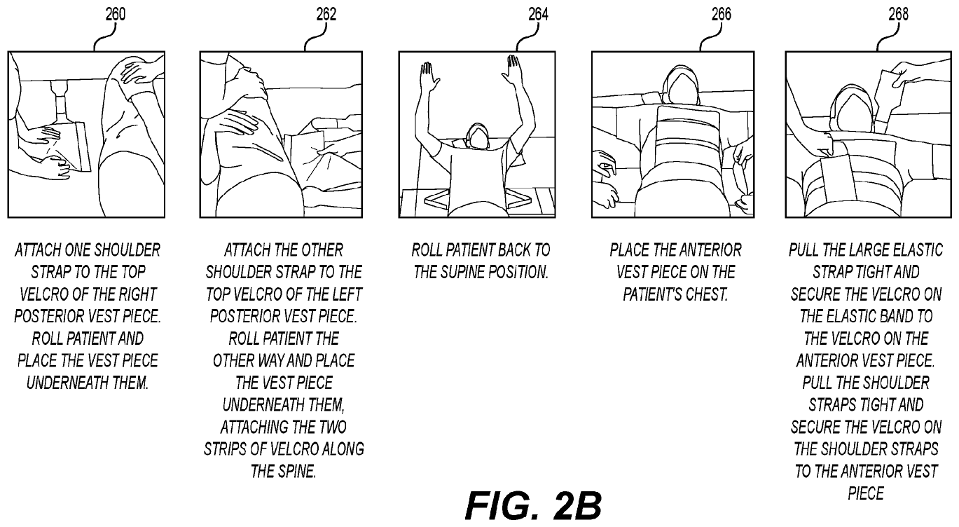

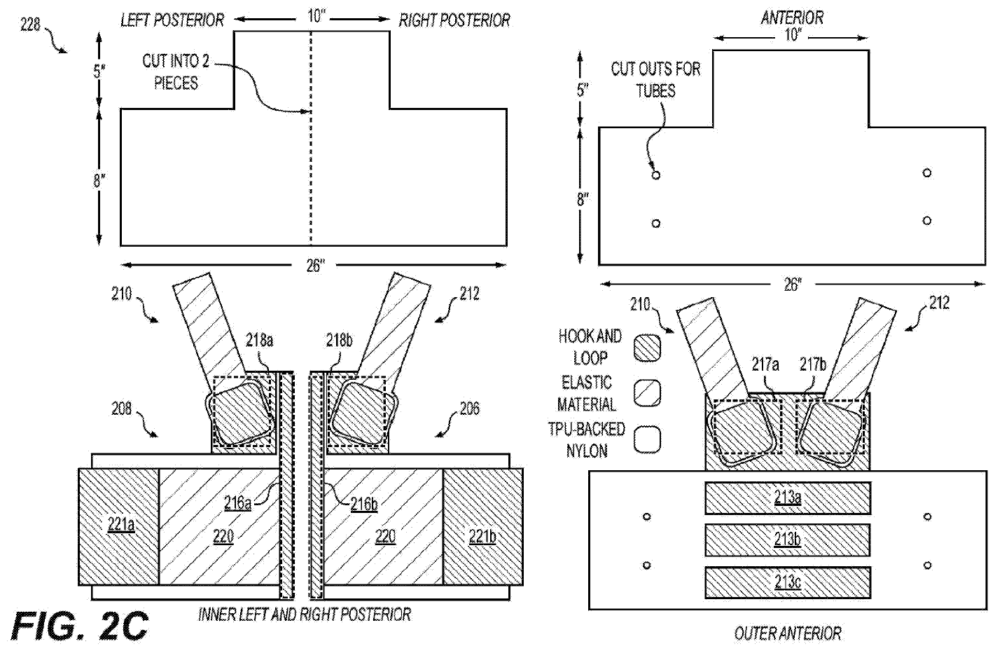

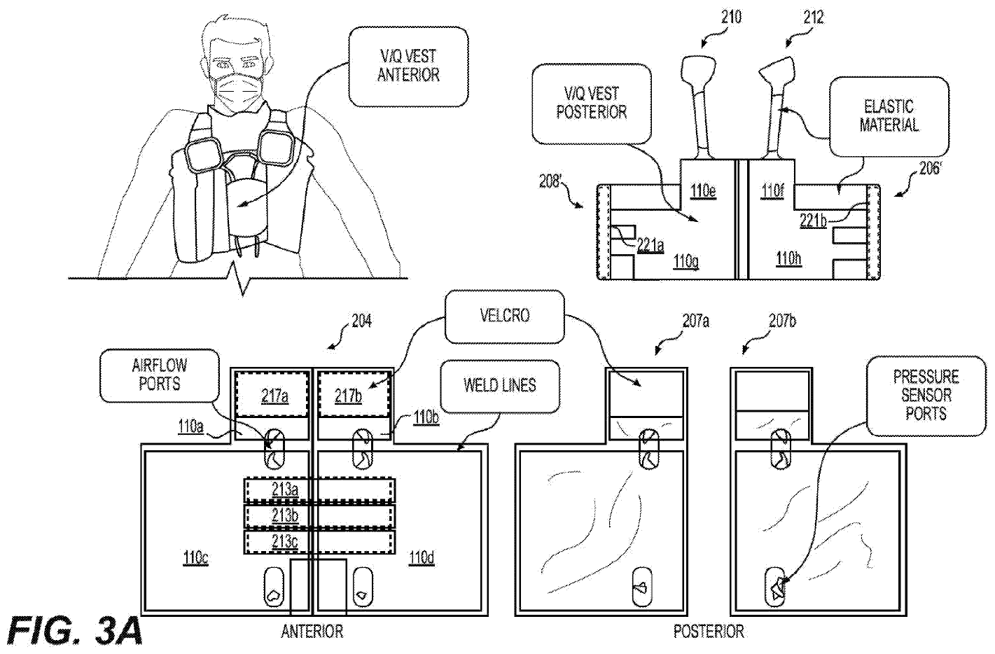

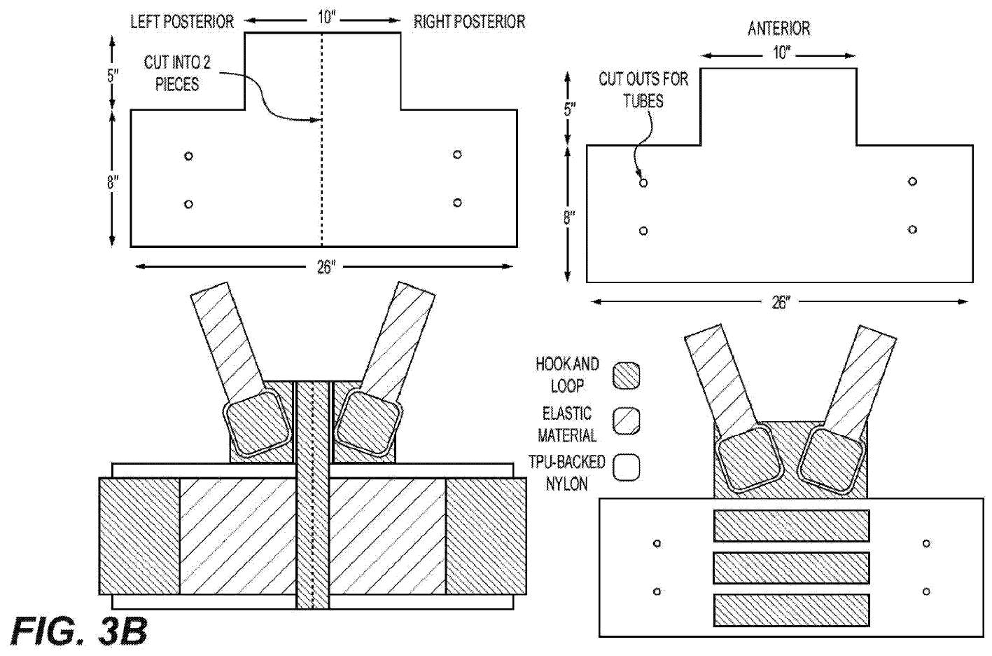

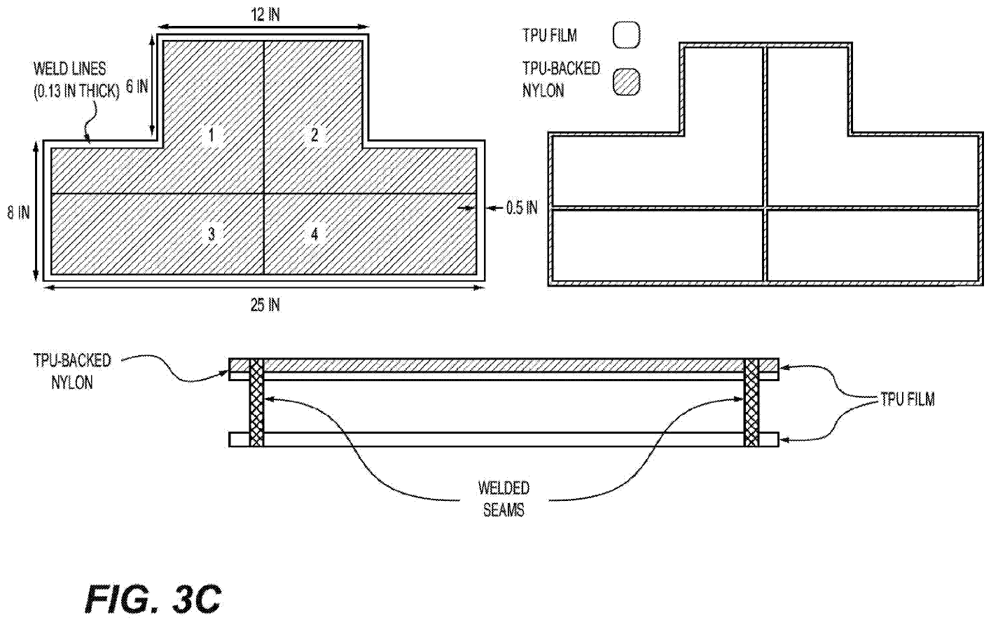

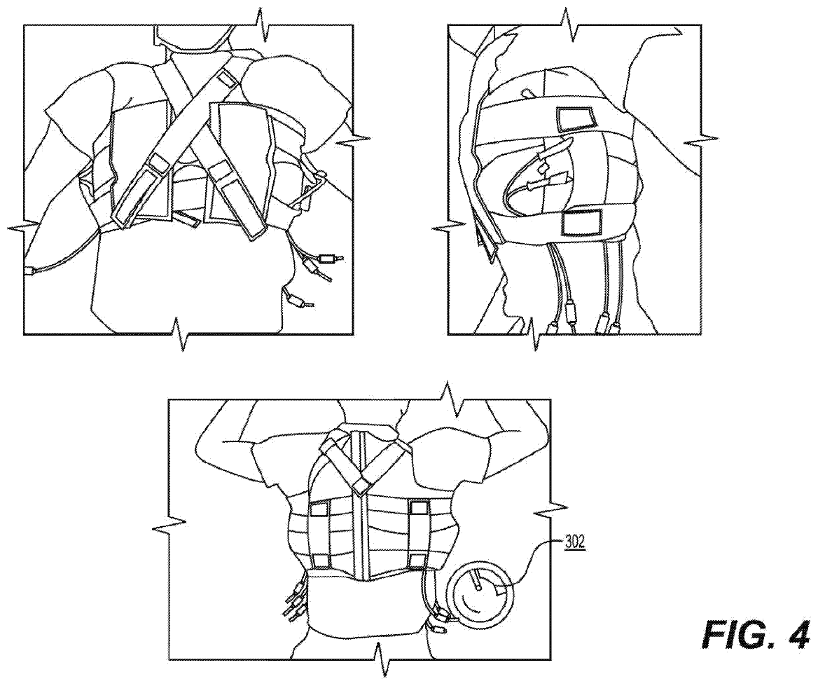

FIGS. 2A-2B are schematic diagrams depicting an example pneumatic compression vest 114 (shown as 202a) according to an illustrative embodiment. FIGS. 3A-3C are schematic diagrams depicting a second example pneumatic compression vest 114 (shown as 202b) according to an illustrative embodiment. FIG. 4 shows a third example pneumatic compression vest 114 (shown as 202c) according to an illustrative embodiment.

Example V/Q Vest #1: FIG. 2A shows an example pneumatic compression vest 114 (shown as 202a) in an assembled and unassembled view. The V/Q vest 202a is designed to mimic the effects of weights being placed on patients' chests. The V/Q vest 202a is form of four main components: an anterior pneumatic component 204, a left and right posterior elastic components 206, 208, and a set of elastic shoulder straps 210, 212.

In the example shown in FIG. 2A, the anterior pneumatic component 204 includes a set of air-tight bladders (e.g., 110a-110d, defined by boundaries 209a-209d) that can be inflated via a set of pneumatic tubes 214 to the desired pressure. This internal pressure then causes the bladders 110 of the vest 202a to expand, and with the restrictive elastic band 220 that is wrapped around the patients, imparting pressure onto the thorax of patients. The anterior pneumatic component 204 in this example is configured to be placed at the anterior of the patient and to form a vest in combination with the posterior elastic components 206, 208, and the set of elastic straps 210, 212. The anterior pneumatic component 204 includes a set of fasteners 213 (shown as 213a, 213b, 213c) to couple with the fasteners 220 of the posterior elastic components 206, 208. The anterior pneumatic component 204 includes a second set of fasteners 217 (shown as 217a, 217b) to couple with the fasteners 220 of the elastic shoulder straps 210, 212.

The anterior pneumatic component 204 can be constructed with a combination of elastic material and thermoplastic polyurethane (TPU), such as a thermoplastic-coated nylon. Each inflatable bladder 110 can be a laser-formed laminar bladder, e.g., formed of a soft/wearable material, fabric, or textile. Each inflatable bladder may be an independently controllable component that is associated with a target location of the patient's body.

Each inflatable bladder may be an independently controllable component per the individual pneumatic tubes 214 that is associated with a target location of the patient's body. In other embodiments, two or more inflatable bladder 110 may be controlled via a pneumatic tube 214. While the example of FIG. 2A shows the number of inflatable bladders 110 of the anterior pneumatic component 204 to be 4, it should be appreciated that the number of inflatable bladder 110 can be 1, 2, 3, 4, 5, 6, 7, or 8. In some embodiments, the number of inflatable bladders 110 is greater than 8. In some embodiments, the left and right posterior elastic components 206, 208 may include inflatable bladders 110 (see FIGS. 3A-3C).

The pneumatic tubes 214 of the anterior pneumatic component 204 are placed outside of the component 204 to strength the bladder by avoiding having interior corners that can form stress points. In some embodiments, the tubes maybe routed within the components.

The left and right posterior elastic components 206, 208, in this example, has an “L” shape in which each includes the restrictive elastic band 220 extending along its horizontal/circumferential length. The left and right posterior elastic components 206, 208, each include a vertical fastening member 216 (shown as 216a, 216b) to couple to one another on one side of the horizontal/circumferential length to form a single elastic component to wrap around the anterior pneumatic component 204. The left and right posterior elastic components 206, 208, each also include a second fastening member 218 (shown as 218a, 218b) to couple to the respective elastic straps 210, 212. The left and right posterior elastic components 206, 208, each also include a third fastening member 221 (shown as 221a, 221b) to couple to one another on the other side of the horizontal/circumferential length.

The elastic shoulder straps 210, and 212 each is an elongated strap that includes an elastic section 222 that terminates with a set of fasteners 224 (shown as 224a, 224b) and 226 (shown as 226a, 226b) on each side.

FIG. 2C shows a diagram of the anterior pneumatic component 204, the left and right posterior elastic components 206, 208, and the set of elastic shoulder straps 210, 212. FIG. 2C shows in diagram 228 the nylon section of the left and right posterior elastic components 206, 208. FIG. 2C also shows in diagram 230 the nylon section of the anterior inflatable component 204.

Example Method for Donning Pneumatic Compression Vest. FIG. 2B shows an example method of donning the pneumatic compression vest (e.g., 202a). At step 260, a first shoulder strap of the pneumatic compression vest is attached to the top Velcro of the right posterior vest piece, and the patient is rolled so that the pneumatic compression vest is positioned underneath them. At step 262, a second shoulder strap of the pneumatic compression vest is attached to the top Velcro of the left posterior vest piece. Then, the patient is rolled in the opposite direction, and the pneumatic compression vest is placed underneath them, attaching the two strips of Velcro along the spine. At step 264, the patient is rolled back to a supine position. At step 266, the anterior vest piece is placed on the patient's chest. At step 268, the large elastic trap is pulled tight, and the Velcro is secured on the elastic band to the Velcro on the anterior vest piece. Then, the shoulder straps are pulled tight to secure the Velcro on the shoulder straps to the anterior vest piece.

Example #2: As noted above, FIGS. 3A-3C are schematic diagrams depicting a second example pneumatic compression vest 114 (shown as 202b) according to an illustrative embodiment. In this example, the V/Q vest 202b is also formed of four main components: an anterior pneumatic component 204, a left and right posterior elastic components 206′, 208′ having a pneumatic component 207 (shown as 207a, 207b), and a set of elastic shoulder straps 210, 212. While the left and right posterior elastic components 206′, 208′ are indeed separate components, in FIG. 3A, they are shown connected to one another in an assembled configuration along with the straps 210, 212 attached thereto.

In this example, the anterior pneumatic component 204 includes 4 inflatable bladders 110a-110d to compress the front/anterior region of the patient's torso and the left and right posterior elastic components 206′, 208′ collectively have 4 inflatable bladders 110e-110h to compress the back/posterior region of the patient. The anterior portion of the pneumatic compression vest 210 can comprise a combination of elastic material and thermoplastic polyurethane (TPU), such as a thermoplastic-coated nylon.

The pneumatic tubes 214 of the anterior pneumatic component 204 and the left and right posterior elastic components 206′, 208′ are placed outside of the component 204 to strength the bladder by avoiding having interior corners that can form stress points.

FIG. 3B shows a diagram of an example configuration of the anterior pneumatic component 204, the left and right posterior elastic components 206′, 208′, and the set of elastic shoulder straps 210, 212. In FIG. 3B, the left and right posterior elastic components 206′, 208′ is not shown with the inflatable bladder 110.

FIG. 3C shows an example construction of the anterior pneumatic component 204. The figures shows the weld line, e.g., formed by an impulse sealer.

Example #3: As noted above, FIG. 4 shows a third example pneumatic compression vest 114 (shown as 202c) according to an illustrative embodiment. The pneumatic compression vest 202c is configured for manual operation and includes a foot-activated manual pump 302 that connects to the.

### Elastic Restriction Elements

The bladders of the vest may be restricted from expanding away from the patient using restriction elements, such as elastic straps. In some examples, the restriction elements/elastic straps are stretched around the patient to the anterior vest piece, where they are secured with Velcro, constricting both the anterior and posterior bladders. This restriction is crucial since the pressure imparted on the patient is proportional to the force produced by the elastic straps when stretched. Since the material properties of the materials used to create the pneumatic vest are unknown, the exact mathematical relationship between the pressure imparted on the patient and the internal pressure of the bladder is unknown. A relationship can be asserted through a static equilibrium analysis of the bladder per Equation 1.

$${P}_{int} {A}_{int} = {P}_{pat} {A}_{pat} + 2 {σ}_{mem} {A}_{mem} \qquad ( Eq . 1 )$$

Since the membrane stress and the cross-sectional area of the membrane are greater than or equal to zero, the relationship can be expressed as Equation 2.

$${P}_{int} {A}_{int} \geq {P}_{pat} {A}_{pat} \qquad ( Eq . 2 )$$

Another property is that both the ratio in areas in Equation 2 and the membrane stress of the bladder are functions of the stiffness of elastic used to constrict the bladders, as shown in Equation 3.

$${lim}_{k \to ∞} \frac{{A}_{pat}}{{A}_{int}} = 1 \& {lim}_{k \to ∞} {σ}_{mem} = 0 , ( h = 0 ) \qquad ( Eq . 3 )$$

Per Equation 3, as the stiffness increases, the ratio of pressure imparted on the patient and the internal pressure of the bladder grow closer together. With higher stiffness, the bladder walls also do not separate as much, causing the membrane stress to diminish to zero.

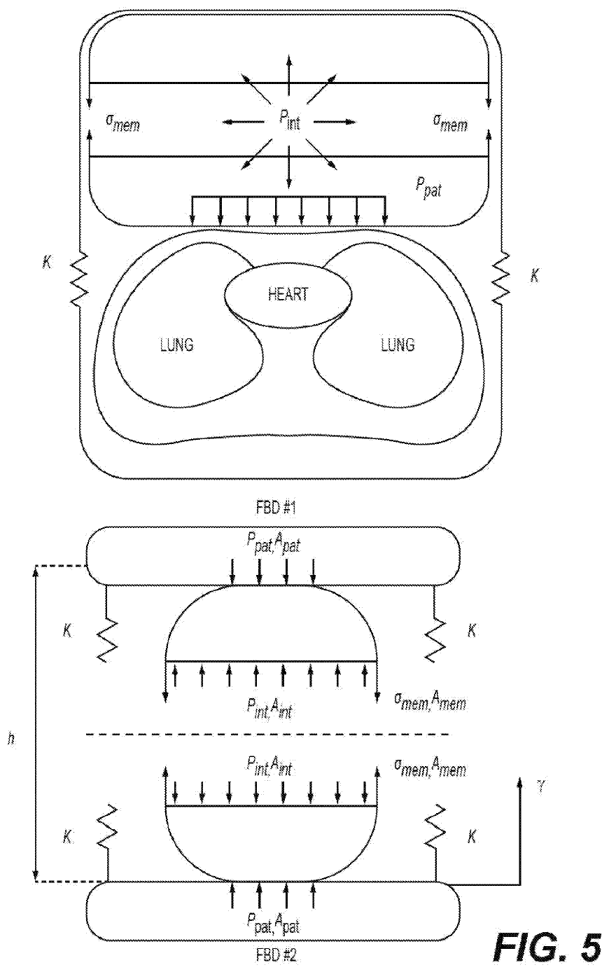

FIG. 5 is a schematic diagram illustrating the effect of using different stiffnesses of elastic to restrict the expansion of the inflatable bladders away from the patient. FIG. 5 shows a free-body diagram of a simplified vest composed of a single bladder. The bladder has been cut as signified by the shaded region. The spring, with stiffness k, signifies the elastic band of the V/Q Vest that restricts the bladders from expanding away from the patient.

As shown, the internal pressure, Pint, remains constant in both scenarios, and k₂>k₁. With higher stiffness elastic, the pressure imparted on the patient is increased and limited to the internal pressure of the bladder. In FIG. 5, the magnitude of the pressure imparted on the patient is signified by the length of the arrows.

### Sensor V/Q Vest Devices

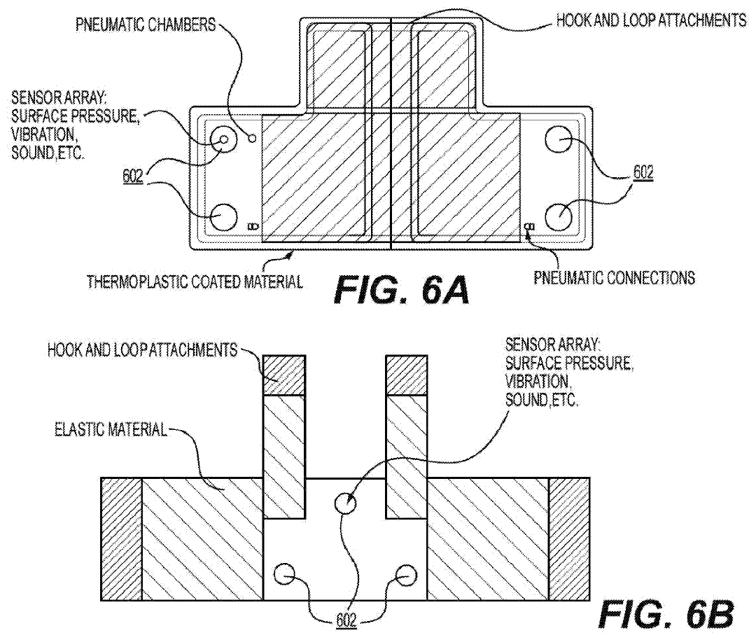

FIGS. 6A and 6B each shows an example configuration of the V/Q vest with sensor arrays 116. FIG. 1B shows a schematic diagram depicting an exemplary pneumatic compression vest system 100 (shown as 100a) with the sensor array 116 for transthoracic manipulation for optimizing oxygenation for a patient 101 in accordance with an exemplary embodiment.

In FIGS. 6A and 6B, the sensors 602 may include a surface pressure sensor(s), vibration sensor(s), sound sensor(s), temperature sensor(s), etc.

As further depicted, the pneumatic compression vest includes at least a first sensor array, a second sensor array, a third sensor array, and a fourth sensor array that are each positioned on the anterior portion of the pneumatic compression vest. Each sensor array may include a pressure sensor, vibration sensor, audio sensor, combinations thereof, and/or the like.

In some implementations, each sensor array 602 is configured to obtain sensor data (e.g., a localized transcutaneous pressure value, cardiovascular pressure values, and/or an esophageal pressure value) from a particular location of the pneumatic compression vest 210 and/or patient's body. In some implementations, each sensor array 602 is configured to monitor local and/or regional pressure between the pneumatic vest and the patient's body. By way of example, an audio sensor can be configured to detect sounds such as Rales, Rhonchi, Stridor, or Wheezing. Additionally, a vibration sensor can be configured to detect vibrations caused by fluid events such as, but not limited to, Stenosis. In some embodiments, each sensor array is configured to monitor an internal pressure value for a respective inflatable bladder.

FIG. 3A shows an example V/Q vest (e.g., 202c) configured with sensor ports for pressure sensing.

### Example Method of Operation

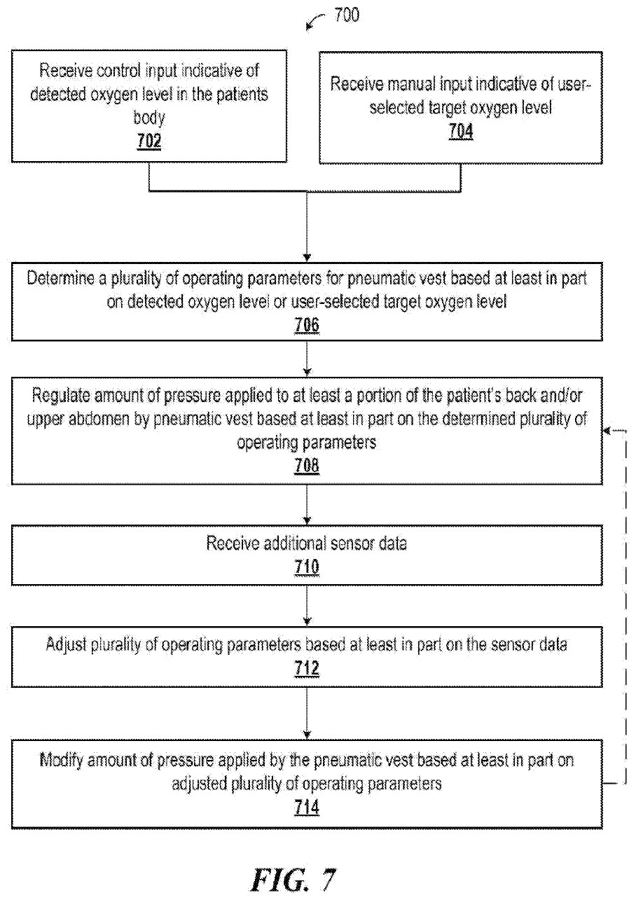

FIG. 7 is a flowchart diagram depicting an example method 700 for improving oxygenation, being either a synergistic mode with proning or replacement therapy.

In some examples, the method 700 may be performed by processing circuitry (for example, but not limited to, an application-specific integrated circuit (ASIC), a central processing unit (CPU)). In some examples, the processing circuitry may be electrically coupled to and/or in electronic communication with other circuitries of the example pneumatic vest, such as, but not limited to, one or more sensor arrays, a memory (such as, for example, random access memory (RAM) for storing computer program instructions), and/or a display circuitry.

In some examples, one or more of the procedures described in FIG. 7 may be embodied by computer program instructions, which may be stored by a memory (such as a non-transitory memory) of a system employing an embodiment of the present disclosure and executed by a processing circuitry (such as a processor) of the system. These computer program instructions may direct the system to function in a particular manner, such that the instructions stored in the memory circuitry produce an article of manufacture, the execution of which implements the function specified in the flow diagram step/operation(s). Further, the system may comprise one or more other circuitries. Various circuitries of the system may be electronically coupled between and/or among each other to transmit and/or receive energy, data, and/or information.

In some examples, embodiments may take the form of a computer program product on a non-transitory computer-readable storage medium storing computer-readable program instruction (e.g., computer software). Any suitable computer-readable storage medium may be utilized, including non-transitory hard disks, CD-ROMs, flash memory, optical storage devices, or magnetic storage devices.

The example method 700 begins at block 702. At block 702, a controller (such as, but not limited to, the controller (e.g., 112) operatively coupled to a pneumatic device 102 described in connection with FIG. 1A or 1B) receives a control input indicative of a detected oxygen level in the patient's body. In some implementations, the control input may be received from a local sensor, another device in communication with the controller, or the like. For example, the controller (e.g., 112) can receive oxygenation data obtained via at least one other device.

Additionally and/or alternatively, at block 704, the controller (e.g., 112) can receive a user-selected target oxygen level. In some embodiments, the user-selected target oxygen level is received via manual input, by adjusting pneumatic vest settings, or via a graphical user interface (GUI).

At block 706, the controller (e.g., 112) can determine a plurality of operating parameters for a pneumatic vest based at least in part on the detected oxygen level, user-selected target oxygen level, and/or received oxygenation data. In some embodiments, the plurality of operating parameters includes a duty cycle for each of a plurality of inflatable bladders of the pneumatic vest. In some implementations, the controller (e.g., 112) can identify a target location of the patient's body (e.g., anterior, posterior, and lateral chest walls) and a corresponding pressure value for the target location. Then, the controller (e.g., 112) can select at least one of the plurality of inflatable bladders associated with the target location and causes the identified inflatable bladder(s) to inflate or deflate to reach the corresponding pressure value.

Then, at block 708, the controller (e.g., 112) can regulate an amount of pressure applied to at least a portion of the patient's anterior, posterior, and lateral chest walls by the pneumatic vest based at least in part on the plurality of operating parameters. For example, the controller (e.g., 112) can control the inflation or deflation of each of a plurality of inflatable bladders based at least in part on the determined plurality of operating parameters. In some implementations, the controller comprises a Proportional-Integral-Derivative (PID) controller that is configured to drive an internal pressure of each of the plurality of inflatable bladders to a specified internal pressure.

At block 710, the controller (e.g., 112) can receive additional sensor data (e.g., vibration data, audio data, pressure values) from at least one sensor array of the pneumatic vest or another monitoring device.

At block 712, the controller (e.g., 112) can adjust the plurality of parameters based at least in part on the received additional sensor data. For example, if the sensor data indicates that the patient has not reached the target oxygen level or that the current oxygen level is below a predetermined threshold, the controller (e.g., 112) can increase the pressure value for the actuated inflatable bladder(s) or actuate one or more additional inflatable bladders. Similarly, if the sensor data indicates that the patient has met or exceeded the target oxygen level or that the current oxygen level is above a predetermined threshold, the controller can reduce the pressure value for the actuated inflatable bladder(s) or de-actuate at least one of the inflatable bladders.

Then, at block 714, the controller (e.g., 112) can modify the amount of pressure being applied by the pneumatic vest (e.g., by actuating one or more additional inflatable bladders or causing one or more inflatable bladders to inflate or deflate to the new pressure value) based at least in part on the adjusted plurality of operating parameters. As depicted in FIG. 4, the controller (e.g., 112) can continuously modify operations of the pneumatic vest in response to receiving new sensor data for the patient as the patient's condition (e.g., oxygenation level) is monitored in real-time.

### Example Control System and User Interface

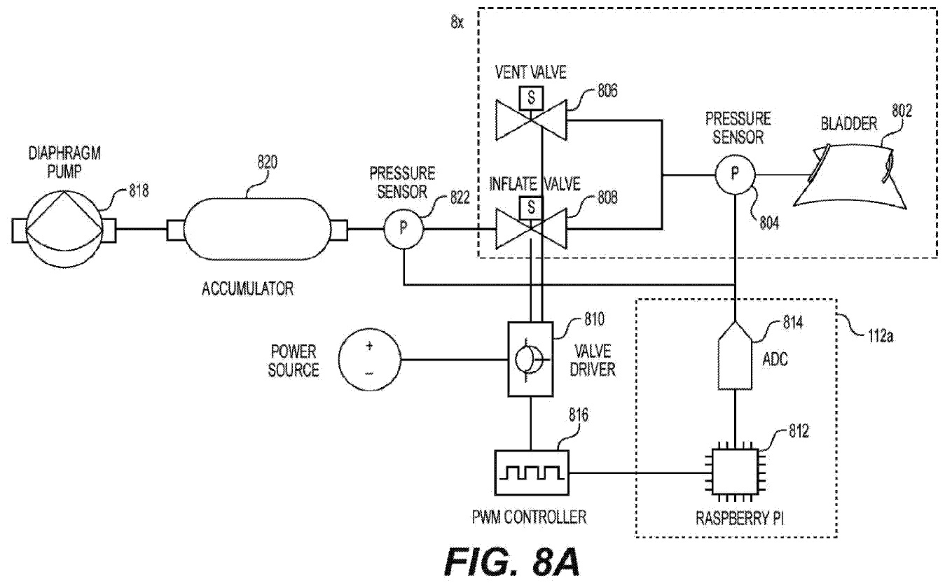

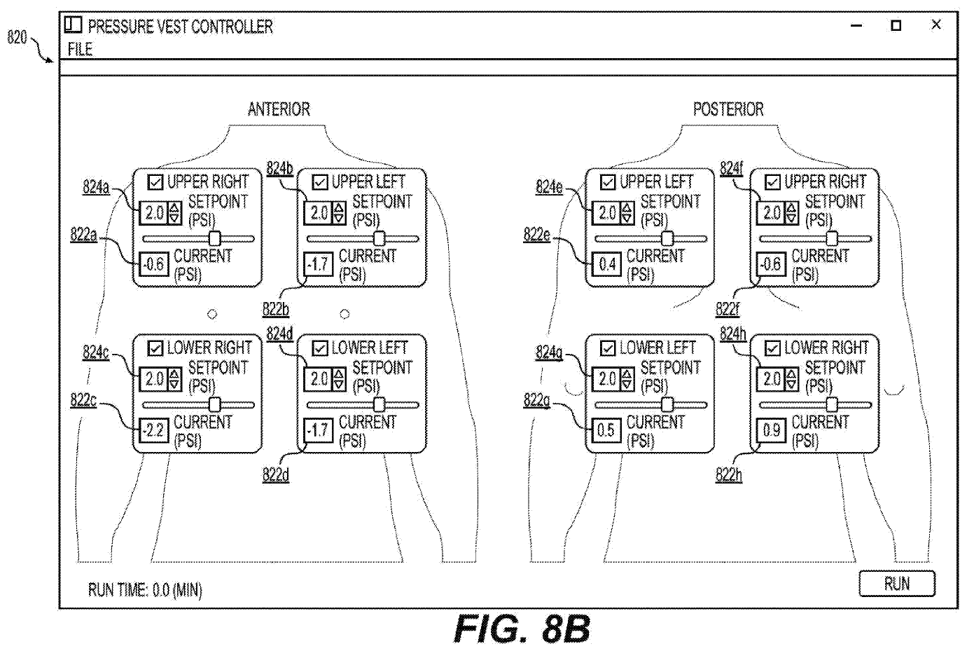

FIG. 8A shows a diagram of an example control architecture for regulating the internal pressure of the independent bladder of the V/Q vest. As shown in the example of FIG. 8A, the individual inflatable bladder 802 of the pneumatic compression vest (e.g., 202a, 202b) is operatively coupled to a first pressure sensor 804, a vent valve 806, and an inflate valve 808. In this example, there are eight sets of the inflatable bladder 802, first pressure sensor 804, vent valve 806, and inflate valve 808.

The internal pressure of the inflatable bladder 802 is electrically controlled via the vent valve 806 and the inflate valve 808 by a valve driver 810. The electronic system includes a controller 112 having a processor 812 (shown as a “Raspberry Pi” 812) and analog-to-digital converter/processing circuitry 814 (shown as “ADC” 814). The processor 812 is configured with instructions to execute the close-loop control based on the sensed pressure provided by the analog-to-digital converter/processing circuitry 814 to drive a PWM controller 816 that then drives the valve driver 810 to drive an internal pressure of the inflatable bladder 802 to a specified or target internal pressure via the valve driver 810 that is operatively coupled to a power source.

The inflate valve 808 is coupled to a pressure source comprising a pump 818 (shown as “Diaphragm Pump” 818), accumulator 820, and a second pressure sensor 822. The second pressure sensor 822 is also operatively coupled to the ADC 814 to determine the fill pressure while the first pressure sensor 804 provides the current pressure reading for the inflatable bladder 802.

The controller (e.g., 112a) may be configured to execute a ventilator mode having preconfigured settings, per manufacturer, to control the mechanical ventilator (e.g., 104) to deliver a tidal volume in a predetermined way. The patient may be a passive recipient of a breath (tidal volume) and may intermittently spontaneously participate in triggering, generating, and sustaining a breath or may do so independently. The level of support may be determined by the clinician and, again may take the form of any combination of rate, volume, pressure, flow, PEEP, and F₁O₂.

Intelligent Ventilator Mode. The mechanical ventilator (e.g., 104) may be configured with “intelligent modes” of operation in which the ventilator is configured to amplify the patient's efforts (to a degree determined by the clinician) based upon measures of patient-generated negative pressure to initiate a breath, rate of breathing, tidal volume, compliance and elastance and overall work of breathing. The control parameter may include pressure, rate, tidal volume, and peak flow, per Table 1.

TABLE 1

| Parameter | Description |
|---|---|
| Pressure | Pressure refers to the driving pressure of the ventilator to |
| | generate a minimum tidal volume in addition to pressure |
| | limits (both maximum and minimum). |
| Rate | Rate refers to a number of breaths per minute delivered by |
| | the ventilator. |
| Tidal volume | Tidal volume refers to the volume delivered by the |
| | ventilator for each breath, or generated by the patient, |
| | which is determined by the clinician and generated by either |
| | volume or pressure control of the ventilator. |
| Peak flow | Peak flow refers to the rate at which the gas is delivered by |
| | the ventilator during inspiration. |
| PEEP | PEEP (positive end expiratory pressure) refers to the |
| | pressure measured at the end of each breath to prevent |
| | alveoli from collapsing, i.e., sufficient pressure to expand |
| | formerly collapsed alveoli in the diseased lung regions in |
| | order to enhance oxygenation. |
| F_{I}O₂ | F_{I}O₂ refers to the fractional concentration of oxygen. For |
| | example, an F_{I}O₂ of 0.5 represents 50% oxygen |
| | concentration. |
| Inspiratory | Inspiratory time refers to the time over which a mandatory |
| time | breath is delivered by the ventilator. |
| Oxygenation | Oxygenation goals may be achieved within a range of |
| goals | clinician-determined patient based physiologic limitations |
| | that may accompany inflation of the vest. These include |
| | inadvertent oxygen desaturation, excessively high lung |
| | pressures associated with vest inflation (peak and plateau), |
| | significant reduction in tidal volume, impairment of |
| | ventilator flow, excessive trans thoracic pressures or any |
| | other evidence of impairment of ventilator function. |
| | Similarly, cardiovascular side effects also may be included, |
| | such as excessive swings in blood pressure (severe |
| | hypo/hypertension) and heart rate. “Vest physiology” will |
| | henceforth refer to all cardiorespiratory effects of the vest, |
| | the patient and mechanics of the ventilator. |

Intelligent VestMode. The controller (e.g., 112a) may be configured with “intelligent modes” of operation for the V/Q vest. When the vest operates in the intelligent mode, the controller (e.g., 112a) may be configured to receive a predetermined target oxygenation set by the clinician or user as determined by clinically relevant measures of patient oxygenation, such as, but not limited to, arterial blood gases, pulse oximetry, etc., which also may be monitored by the ventilator or input/uploaded into the vest/ventilator interface/algorithm.

The clinician may program, in the intelligent mode, the vest to incrementally inflate during full cardiorespiratory monitoring in order to reach oxygenation goals as long as preset physiologic limits of ventilator mechanics are not exceeded. As noted above, these physiologic limits include all aspects of cardiorespiratory physiology as monitored by the ventilator, input/uploaded into the ventilator, and patient physiologic parameters as part of the ICU standard of care.

In some embodiments, in the intelligent mode, the clinician may program the vest to continually evaluate oxygenation (on a regular time interval that can be set by the clinician) and the need to either inflate the vest should oxygen measure decline below goals or alternately evaluate the impact of deflation should oxygenation levels exceed goals.

In some embodiments, in the intelligent mode, the clinician may program the vest to deflate by increments determined by the clinician should oxygenation levels exceed goals. Both oxygenation and vest physiology may continue to be monitored by conventional means and via the ventilator. If deflation causes desaturation, reinflation may be reinstituted until oxygenation goals are achieved again. The pressure point of desaturation may be recorded and may serve as a “marker” for the vest/ventilator system. The vest may, for example, only deflate to 10% psi above that previous level in order to prevent desaturation.

Graphical User Interface. FIG. 8B shows an example graphic user interface 820 for an electro-pneumatic controller, e.g., generated by a controller. As shown in the example of FIG. 8B, the GUI may include a current measurement reading 822 (shown as 822a-882h) for each of the inflatable bladders 802, e.g., via pressure sensor 804. The GUI 820 also includes inflatable pressure settings 824 (shown as 824a-824h) for each of the inflatable bladders 802. In this example, the GUI 820 provides 8 controllable setpoints for 8 inflatable bladders. The number of controllable setpoints can be established based on the number of inflatable bladders. In some embodiments, the setpoints can be aggregated, in which the setpoints can be set for two or more inflatable bladders.

### Experimental Results and Additional Examples

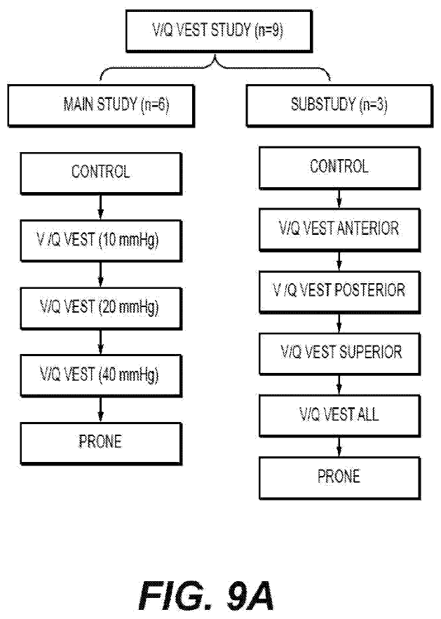

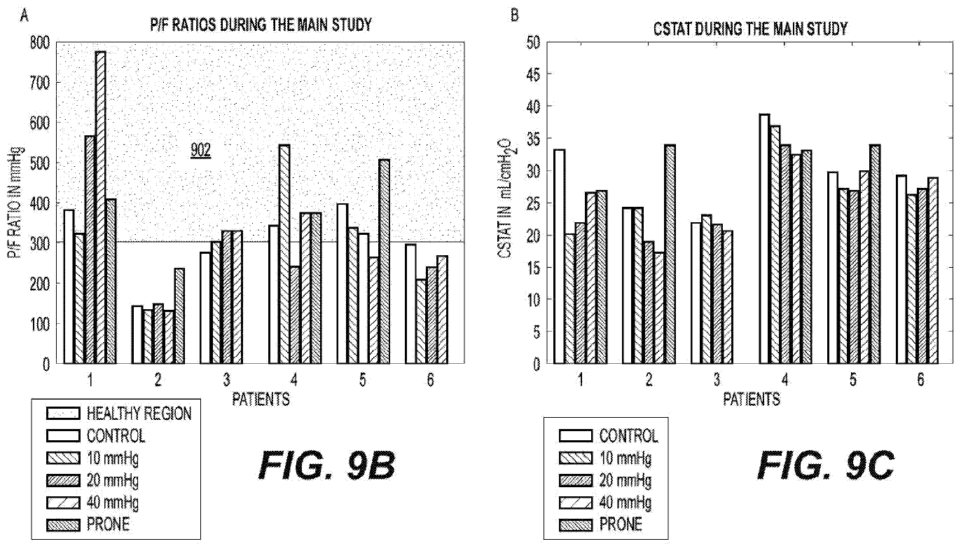

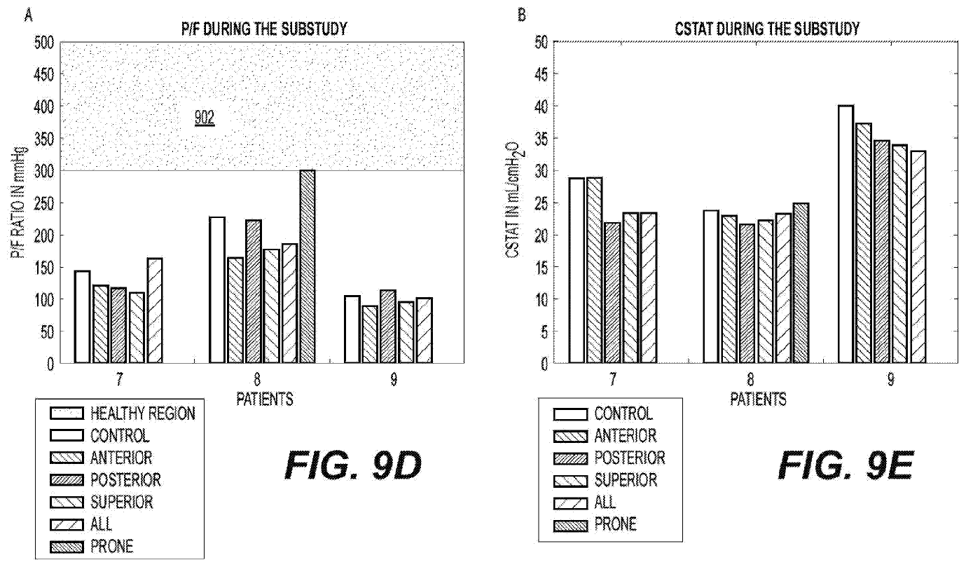

A series of studies have been conducted to develop and evaluate pneumatic compression vest system for transthoracic manipulation for optimizing oxygenation for a patient. A first study was conducted that evaluated two prototype systems on nine patients with ARDS from Coronavirus disease 2019 (COVID-19). The first study included a main sub-study (n=6) and the second sub-study (n=3). FIG. 9A shows an overview of the study flow. The goal of the main study was primarily to see if the V/Q Vest with all the bladders inflated to the same pressure could: (a) improve patients' condition similarly to proning or other treatments, (b) be used to predict whether patients would respond well to proning, and (c) decrease patients' static lung compliance. After the first main study and sub-study with the electro-pneumatically controlled vests, a third prototype was developed that was configured to be more robust and could be manufactured on a larger scale.

The first study observed the prototype systems and associated V/Q Vest treatment provided an average increased in oxygenation for all patients by 19.7±38.1%. Six of the nine patients responded positively to the V/Q Vest treatment, exhibiting increased oxygenation. The V/Q Vest also helped hospital staff predict that three of the five patients that were proned would experience an increase in oxygenation. An increase in oxygenation resulting from V/Q Vest treatment exceeded that of the proning treatment in two of these five proned patients.

### Methodology.

First Main Study. The first phase of the main study was to establish the control for all six of the patients. All patients were placed in the supine position and on mechanical ventilation. Their vitals were recorded for an hour after their vitals stabilized. During the second through the fourth phase of the main study, patients 1-6 had the V/Q Vest applied while still on mechanical ventilation. The hospital staff increased the V/Q Vest pressure between these three 1-hour-long trials (10 mmHg for the first hour, 20 mmHg for the second hour, and 40 mmHg for the third hour). Every bladder of the V/Q Vest in this main study was inflated to the same pressure. At the end of each hour, their vitals were recorded before engaging in the next V/Q Vest treatment pressure. The last phase of the main study was to remove the V/Q Vest from the patient and prone the patient while still on mechanical ventilation only if proning was clinically feasible. Patients 3, 4, 7, and 9 were not able to be safely proned due to clinical reasons or were not proned since proning was done and minimal effects were seen.

Second Sub-Study. The sub-study was performed to determine how the location of the V/Q Vest pressure affects: (a) patients' response, and (b) patients' static lung compliance. Patients 7-9 were subjects in this sub-study. This sub-study had the same control and proning phase as the main study. The middle four phases used the V/Q Vest to apply pressure to specific locations of the patient while still on mechanical ventilation. For the first location, only the anterior bladders were inflated to 30 mmHg. The second was only inflating the posterior bladders to 30 mmHg. The third was only inflating the small anterior- and posterior-superior chambers to 40 mmHg. For the last location permutation, all the bladders were inflated to 30 mmHg.

Third Study. The first step of the study protocol was to measure the patients' vitals while sedated in the supine position to be used as controls for the study. The V/Q Vest was then donned on the participants, and all the bladders were inflated to the first internal pressure. The pressure of the V//Q Vest was inflated to be varied by patient depending on their response. The participants were then given an hour for their vitals to stabilize after which their vitals were recorded again. Then the V/Q Vest was inflated to the next preset value, and this process repeated. After 3 hours of increasing the V/Q Vest pressures, the vest was doffed from the participant, and they were proned according to the Emory University Hospital protocol. After an hour in the prone position, their vitals were recorded, concluding the study. Two participants, patient #3 and patient #4 were not able to be safely proned due to complications. For these two participants, the study concluded after the third hour of increasing pressure applied by the V/Q Vest.

The inclusion criteria for the first study were patients with age >18 years, presentation of ARDS due to COVID-19, currently intubated patients on ventilator support, and authorized representative's ability to provide informed consent. The presentation of ARDS was defined by the 2012 Berlin Criteria and rated in severity by patients' P/F ratios. Nine patients with ARDS caused by COVID-19 gave informed consent to participate in this study. All the patients were admitted to the Acute Respiratory Intensive Care Unit. Table 2A shows the relevant demographics of the nine patients in the first study.

TABLE 2A

| | | | Weight | | |
|---|---|---|---|---|---|
| Patient | Gender | Age | (kg) | BMI | V/Q Vest Iteration |
| 1 | Male | 72 | 73 | 27.4 | 1 |
| 2 | Female | 48 | 154 | 38.1 | 1 |
| 3 | Male | 60 | 61 | 20.6 | 1 |
| 4 | Male | 65 | 79 | N/A | 1 |
| 5 | Male | 89 | 50 | 28.5 | 1 |
| 6 | Male | 67 | 102 | 31.0 | 1 |
| 7 | Male | 37 | 116 | 34.7 | 2 |
| 8 | Male | 50 | 78 | 22.9 | 2 |
| 9 | Male | 77 | 128 | 39.8 | 2 |

In the first study, all patients were on mechanical ventilators, which were programmed by hospital staff to optimize each patient while in the supine position without the V/Q Vest applied. The mechanical ventilators held the fraction of inspired oxygen (FiO₂), tidal volume (V_{tid}), and positive end-expiratory pressure (PEEP) constant for every patient throughout the study. The relevant vital information recorded from the mechanical ventilators and analyzed in this work were PaO₂, FiO₂, and Cₛₜₐₜ. Static lung compliance (Cₛₜₐₜ) is calculated by the following equation.

$${C}_{stat} = \frac{Δ V}{Δ P} = \frac{{V}_{tid}}{{P}_{plt} - PEEP} \qquad ( 5 )$$

Since the tidal volume and PEEP were controlled by the mechanical ventilator, the only effect on Cₛₜₐₜ was deemed to come from the plateau pressure. It was expected that the vest treatment or proning would decrease lung compliance by stiffening the thorax expansion of patients. P/F ratios disseminated here were calculated by simply dividing PaO₂ by FiO₂. It was observed that all nine patients had FiO₂ greater than 0.5.

Table 2B shows the demographics of the six patients in the second study.

TABLE 2B

| Patient | | | Weight | |
|---|---|---|---|---|
| ID | Gender | Age | (kg) | BMI |
| 1 | Male | 72 | 73 | 27.4 |
| 2 | Female | 48 | 154 | 38.1 |
| 3 | Male | 60 | 61 | 20.6 |
| 4 | Male | 65 | 79 | N/A |
| 5 | Male | 89 | 50 | 28.5 |
| 6 | Male | 67 | 102 | 31 |

Prototype Systems. Two prototype systems were developed and employed in the study. The first iteration of the V/Q Vest was used only in the main study. The second iteration of the V/Q Vest was used in the sub-study.

First V/Q Vest Design. The first iteration of the V/Q Vest had eight independent bladders, and for the main study, these eight bladders were always inflated to the same pressure. This iteration of the V/Q Vest had issues with the welds tearing around the interior corners of the anterior and posterior bladders. As a result, the V/Q Vest treatment was immediately stopped, and these patients' data were excluded from the analysis. We also observed that the lower bladders on the anterior of the vest were not applying as much pressure as the upper bladders. This is thought to happen because the cross-sectional area of the lower bladders is much smaller. These two issues with the first iteration of the V/Q Vest are why the design was modified.

An electro-pneumatic controller was fabricated to autonomously regulate the internal pressure of the independent bladders that form the V/Q Vest. The controller senses and regulates the internal pressure of all the bladders at 100 Hz. The pressure of each bladder was controlled using two solenoid valves in series, one for inflation and one for deflation. A Proportional-Integral-Derivative (PID) controller was implemented to drive the internal pressure of the bladders to a specified internal pressure set by the hospital staff A GUI was developed to simplify the control of the V/Q Vest for the hospital staff. The GUI allowed the hospital staff to independently control each bladder and monitor the internal pressure of the V/Q Vest in real-time. For further safety of the patient, an emergency stop was implemented that cuts power to the controller and vents all the bladders leading to rapid deflation.

Second V/Q Vest Design. The second iteration of the V/Q Vest only had four independent bladders. This iteration of the V/Q Vest still had upper and lower chambers on both the anterior and posterior of the vest, but the upper and lower chambers were connected, resulting in only two independent bladders on both the anterior and posterior of the V/Q Vest. This design was instituted to avoid interior corners that caused failures in the first iteration of the V/Q Vest. FIG. 3A-3C shows the second iteration of the V/Q vest. The second iteration of the V/Q Vest used an electro-pneumatic controller. Only four of the eight electro-pneumatic control systems were used, and an updated GUI was implemented. The second iteration also had separate pneumatic connectors for the pressure sensors. It was observed from the first iteration that having the pressure sensors connected to the tubes that inflated and deflated the bladders resulted in pressure instabilities. As airflow passed by the tubes connected to the pressure sensors, the static pressure measured by the sensors dropped, causing instabilities in the PID control algorithm. These extra connectors were implemented to minimize the airflow past the pressure sensors so that they would more precisely sense the static pressure of the bladders of the V/Q Vest.

Third V/Q Vest Design. After the first main study and sub-study with the electro-pneumatically controlled vests, a third prototype was developed that was configured to be more robust and could be manufactured on a larger scale. The third prototype included only an anterior portion to improve manufacturability and reduce cost. The first sub-study illustrated no clear benefit when applying pressure to only the anterior of the patient or to the anterior and the posterior of the patient. The posterior ribs are more rigid than the anterior ribs, so it is thought that this pressure imparted on the posterior has negligible effects on the patient. In the third prototype, the electro-pneumatic controller was replaced with a modified sphygmomanometers (blood pressure cuff devices) that is calibrated to measure pressures between 10 mmHg and 300 mmHg with a precision of ±5 mmHg [27]. The third prototype was also manufactured using a radio frequency (RF) welding process. FIG. 2A-CC shows an example of the third iteration of the V/Q vest.

Benchtop Evaluation of Third V/Q Vest Design. To establish the amount of pressure the V/Q Vest needs to apply to patients that experience symptoms of ARDS, 5 L saline bags were placed on top of patients' chests while in the supine position. Their oxygenation level was recorded for an hour. It was found that 2 bags stacked on top of each other applied enough pressure to the patient to see significant increases in oxygenation. A clinically significant increase in PaO2 of the P/F ratio is around 20%. The pressure imparted on the patients from the weight of the two 5 L saline bags is roughly equivalent to 20 mmHg. Hence, the V/Q Vest needed to apply at least 20 mmHg of pressure to patients' thorax. Since the internal pressure of the bladders is always higher than the pressure imparted to the user, the V/Q vest was designed to withstand 100 mmHg of internal pressure as a factor of safety. Further testing characterized the leak rate of the bladders to ensure that the controller could maintain the desired internal pressure. Three test bladders were inflated to 100 mmHg and the pressure over 2 hours was recorded to establish the leak-rate of the bladders. An exponential decay model was fit to the pressure vs time data. After many iterations of the bladders, the final bladder design presented in this work does not leak more than 1 mmHg per minute. This leak rate of these bladders is lower than the rate of inflation from the electro-pneumatic controller so the controller can still maintain the desired pressure.

Results of Main Study using the First V Q Vest. FIG. 9B shows P/F ratios of patients 1-6 throughout the main study, and FIG. 9C shows static lung compliance of patients 1-6 throughout the main study. The region 902 in FIG. 9B denotes the healthy P/F ratio region.

Table 3 shows the patients' FiO₂ and PaO₂ levels in mmHg during each phase of the main study. Patients' FiO₂ was held constant throughout every trial. Four of the six patients exhibited an increase in PaO₂ levels while wearing the V/Q Vest. It is surmised that patient 2 did not see an increase in PaO₂ levels from the V/Q Vest due to a high BMI or excess soft tissue. It is hypothesized that excess thoracic tissue disperses the pressure applied to the chest wall away from the lungs.

TABLE 3

| | | | V/Q (10 | V/Q (20 | V/Q (40 | |
|---|---|---|---|---|---|---|
| Patient | FiO₂ | Control | mmHg) | mmHg) | mmHg) | Prone |
| 1 | 0.5 | 95 | 81 | 140 | 193 | 102 |
| 2 | 1 | 141 | 130 | 143 | 129 | 236 |
| 3 | 0.5 | 68 | 76 | 82 | 82 | N/A |
| 4 | 0.55 | 103 | 164 | 73 | 112 | 112 |
| 5 | 0.5 | 99 | 84 | 81 | 66 | 126 |
| 6 | 0.6 | 106 | 74 | 86 | 96 | N/A |

While Pao₂ is a good measure to ensure that patients are getting the oxygenation needed to remain in stable condition, this measure alone may not necessarily reflect the patients' efficiency at infusing oxygen from their environment into their bloodstream, so the P/F ratio is analyzed.

As noted above, FIG. 9B shows the P/F ratios for each patient in the main study. Healthy P/F ratios are between 400-500 at sea level on atmospheric FO₂. The P/F ratios for all but two participants were observed to increase while wearing the V/Q Vest. For patients 5 and 6, proning was more effective at raising their P/F ratios than wearing the V/Q Vest. Patient 2 was morbidly obese and patient 5 had an extensive number of comorbidities. These factors are hypothesized to have led to the decreased effectiveness of the V/Q Vests.

A correlation between the V/Q Vest pressure and the change in P/F ratio compared to the control trial was analyzed with a linear correlation. The resulting correlation coefficient was found to be 0.94 (p=0.22), indicating that the correlation between the V/Q vest pressure and the increase in patients' P/F ratio is uncertain. This result is accepted since there is evident inter-subject variability due to varying severity of ARDS and comorbidities. However, comparing each participant on their own shows that four of the six patients experienced increases in P/F ratios due to the V/Q Vest. In two out of these four patients, the increase in P/F ratios due to the V/Q Vest treatment was higher than their P/F ratios experienced while prone.

The last metric analyzed in this study was the static lung compliance of the patients. FIG. 9C shows the static lung compliance of patients 1-6. Static lung compliance is a measure for determining the sensitivity of the lung to alterations in pressure and is a factor in how hard the person must breathe under their own power. This metric has also been correlated with ARDS diagnosis in both COVID-19 and non-COVID-19 patients with little differentiation. Average lung compliance for patients with ARDS is found to be less than 50 mL/cmH₂O and a healthy adult would have static lung compliance above 150 mL/cmH₂O [24, 25]. All the patients in the main study were observed to have static lung compliances of less than 50 mL/cmH₂O. With the V/Q Vest applied, static lung compliance was observed to decrease depending on the amount of pressure applied to the patients' chest wall only for a few patients. While no correlation was found between V/Q Vest pressure and static lung compliance when grouping all patients together, when comparing each patient individually, the V/Q Vest (inflated to at least one of the three pressures used in this study) was observed to decrease the static lung compliance for all patients. The pressure required to reduce static lung compliance varied between participants which may be why a correlation between static lung compliance and vest pressure was not found when grouping all patients together.

Because the mechanical ventilation was controlled to keep the tidal volume and PEEP constant for each patient throughout the study, it was contemplated that decreasing lung compliance using the V/Q Vest, proning, or placing weights on patients' chest causes patients' plateau pressure to increase. Typically, this increase in plateau pressure would increase alveolus distension, but with the V/Q Vest, the alveolus overdistension is likely reduced due to the pressure the vest imparts on patients' thoracic cavities. Therefore, it is contemplated that the V/Q Vest may also be useful in reducing the risk of lung injury that is caused by an increase of driving pressure from mechanical ventilation [26].

Results of Sub-Study using the Second V Q Vest. FIG. 9D shows the P/F ratios of patients 7-9 throughout the sub-study, and FIG. 9E shows static lung compliance of patients 7-9 throughout the sub-study. The region 902 in FIG. 9D denotes the healthy P/F ratio region.

Table 4 shows the patients' FiO₂ and PaO₂ levels in mmHg during each phase of the sub-study. Patients' FiO₂ was held constant throughout every trial. Two of the three patients experienced an increase in PaO₂ and P/F ratios while wearing the V/Q Vest. Patients 7 and 9 were proned before the study with no apparent response, but the V/Q Vest was successful in increasing their P/F ratios. Patient 8 was able to be proned, and their increase in P/F ratio while proned was higher compared to the V/Q Vest treatment. For patient 8, the V/Q Vest did not help the hospital staff correctly predict the response from the patient while proned.

TABLE 4

| | | | V/Q | V/Q | V/Q | V/Q | |
|---|---|---|---|---|---|---|---|
| Patient | FiO₂ | Control | Anterior | Posterior | Superior | All | Prone |
| 1 | 0.8 | 93 | 78 | 75 | 70 | 105 | N/A |
| 2 | 0.7 | 112 | 81 | 109 | 87 | 92 | 147 |
| 3 | 0.8 | 67 | 58 | 72 | 61 | 65 | N/A |

Table 5 shows the changes in the P/F ratios compared to the control trial with respect to where pressure was applied. No evident correlation was apparent in this small sample size sub-study. However, the sub-study did concur with the evidence from the main study that static lung compliance was decreased during the V/Q Vest treatment. Only patient 7 did not experience a decrease in static lung compliance for anterior V/Q Vest treatment.

TABLE 5

| Patient | ΔP/F Anterior | ΔP/F Posterior | ΔP/F Superior | ΔP/F All |
|---|---|---|---|---|
| 1 | −23 | −28 | −36 | 19 |
| 2 | −62 | −6 | −51 | −41 |
| 3 | −14 | 8 | −9 | −3 |

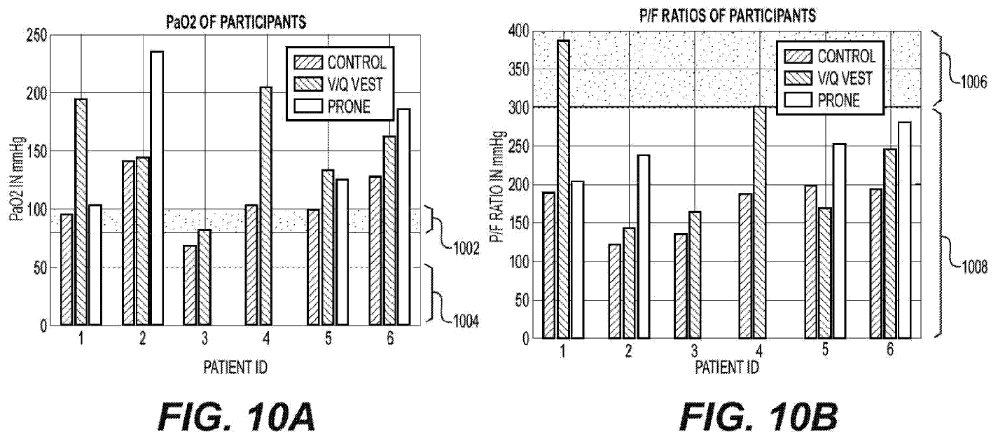

Results of Third Study using the Third V Q Vest. FIG. 10A shows the stead-state PaO2 measurements from the patients during the study. FIG. 10B shows the P/F ratio (PaO2/FiO2) of the patients from the control trial and the measured trial while wearing the V/Q vest.

Specifically, FIG. 10A shows the PaO2 values from the 6 participants in the study. The PaO2 values are shown with the normal range of 80-100 mmHg (see 1002) and hypoxemia range of less than 50 mmHg (see 1004). In FIG. 10B, the P/F ratio values are shown with the normal range of above 300 mmHg (see 1006) and ARDS range of less than 300 mmHg (see 1008).

The V/Q Vest data reported in FIG. 10A are the PaO2 levels that were the highest for the three pressure levels the vest was inflated to. All but one participant exhibited a remarkable increase in PaO2 levels while wearing the V/Q Vest. It is surmised that patient #2 did not see a remarkable increase in PaO2 levels from the V/Q Vest due to a high BMI. Excess tissue may disperse the pressure applied to the chest wall.

Table 6 shows the participant's ΔPaO2-Vest, OVP, and ΔPaO2-Prone values. ΔPaO2-Vest signifies the greatest steady-state change in PaO2 levels from wearing the V/Q Vest compared to the control trial. ΔPaO2-OVP indicates the internal pressure of the bladders on the vest that produce the greatest increase in PaO2 levels for each participant. ΔPaO2-Prone signifies the steady-state change in PaO2 levels found from proning compared to the control trial.

TABLE 6

| Patient | ΔPaO2-Vest | OVP | ΔPaO2-Prone |
|---|---|---|---|
| ID | (mmHg) | (mmHg) | (mmHg) |
| 1 | 98 | 41 | 7 |
| 2 | 2 | 21 | 95 |
| 3 | 14 | 41 | N/A |
| 4 | 101 | 72 | N/A |
| 5 | 34 | 52 | 27 |
| 6 | 35 | 52 | 58 |

From Table 6, it can be observed that all participants were exhibiting symptoms of ARDS since their control P/F ratios were less than 300 while on mechanical ventilation. Normal P/F ratios are between 400-500 at sea level. The P/F ratios for all but two participants increased while wearing the V/Q Vest. For patient #5 and patient #6, proning was more effective at raising their P/F ratios than wearing the V/Q Vest. Patient #2 was morbidly obese, and patient #5 had a pulmonary embolism (PE). These factors are highly suspected of having decreased the effectiveness of the V/Q Vests.

It can be observed from the third study that the V/Q Vest remarkably improved oxygenation and improved hypoxemia among 5 out of the 6 patients who participated in this study. Two patients had higher PaO2 levels with the V/Q Vest compared to proning. Conversely, two patients showed lower PaO2 levels with the V/Q Vest compared to proning. The remaining 2 patients were not able to be proned; however, the V/Q Vest was able to be used and increased these patients' PaO2 levels remarkably. More clinical testing will need to be conducted to clearly differentiate the differences in effects of the V/Q Vest and proning. The V/Q Vest can help improve oxygenation for patients that cannot be safely proned, which could decrease the mortality rate of ARDS.

It is contemplated that the V/Q Vest can improve oxygenation in both male and female patients to the same degree. With the current shape of the vest, excess tissue disperses the pressure imparted on the user's chest wall, which caused less of an impact seen in female patients and obese patients. The V/Q Vest can designed to accommodate the presence of breast tissue in female and morbidly obese patients.

The vest controller can be integrated with the mechanical ventilation and patient monitoring systems to automate the V/Q Vest system to alleviate the workload of the hospital staff needed to treat the symptoms of ARDS.

### DISCUSSION

Critical care patients who experience acute respiratory distress syndrome, or other respiratory diseases or conditions, are commonly placed on mechanical ventilators to improve oxygen delivery and overall gas exchange of the pulmonary system. With the pulmonary inflammation typically accompanying acute respiratory distress syndrome (ARDS), patients can experience significant alterations in ventilation-perfusion ratios resulting in lower blood oxygenation. In these cases, patients are typically rotated, i.e., moved, into a prone position to facilitate improved blood flow to portions of the lung that were not previously participating in the gas exchange process.

Mechanical ventilator therapy is initiated in the setting of lung failure as characterized by either a failure to ventilate or oxygenate or both. The former is characterized by a pathological rise in levels of arterial carbon dioxide, while the latter is associated with abnormally low levels of oxygen which impair cellular function. Both conditions are associated with potentially dangerous physiologic sequelae. While there are many conditions which may lead to lung failure, the most common pathophysiological phenomenon leading to these scenarios is described as ventilation perfusion mismatch (V/Q mismatch). V/Q mismatch occurs when there is an “uncoupling” between the passage of gas through the alveolar wall and uptake/exchange with the circulation. This may be seen with both airway and alveolar pathology where gas does not reach the alveolus or intrinsic alveolar injury prevents its participation in gas exchange. Additionally, lung capillary abnormalities, such as COVID induced embolism or thrombosis (“perfusion”) prevents uptake and exchange of gases as the alveolus is now “uncoupled” from the circulatory system.

Clinicians will then turn to mechanical ventilators which provide numerous modes of respiratory support using combinations of fixed/variable breathing rates, pressure, tidal volume, air flow, and positive end expiratory pressure, among others, to optimize CO₂ and O₂ levels in the patient's body. The results of these interventions are measured through frequent arterial blood gas (ABG) sampling. Simultaneous noninvasive measures are taken via pulse oximetry, which determines the amount of oxygen bound to a patient's hemoglobin, and which will be used in normal cellular processes.

Upon review of the above information, physicians will titrate the parameters of the ventilator to optimize therapeutic interventions. For example, the introduction and manipulation of pressure delivered by the ventilator at the end of exhalation, known as PEEP (positive end-expiratory pressure), will reinflate alveoli that were previously collapsed to be “recruited” once more into the role of gas exchange. By contrast, CO₂ removal is achieved via optimizing the volume of gas delivered with each breath (the tidal volume) and the rate of breaths per minute. Efforts are made to restore oxygenation and ventilation within physiologic limits that do not predispose patients to ventilator-associated lung injury, potentially caused by excessive pressure or volume.

Failure to respond to traditional methods has led to novel and more aggressive approaches, such as placing the patient on their abdomen (proning). Proning is the process of turning a patient with precise, safe motions from their back onto their abdomen (stomach) so the individual is lying face down. While beneficial, proning a patient can carry significant risks: for example, airway obstruction, dislodgement of the endotracheal tube, pressure-related skin injuries, facial and airway edema (swelling), hypotension (low blood pressure), and arrhythmias (irregular heartbeat/rate). The process additionally requires several hospital staff members, e.g., 6-7 staff members, to carry out and does not guarantee an improvement in the patient's condition.

In the event that mechanical ventilatory support fails to adequately oxygenate the patient, then extracorporeal membrane oxygenation (ECMO) may be considered. However, these approaches are not benign and are associated with additional morbidity of 31% at 60 days. See Schmidt M, Hajage D, Lebreton G, et al.; Groupe de Recherche Clinique en REanimation et Soins intensifs du Patient en Insuffisance Respiratoire aiguE (GRC-RESPIRE) Sorbonne Universite; Paris-Sorbonne ECMO-COVID investigators. Extracorporeal membrane oxygenation for severe acute respiratory distress syndrome associated with COVID-19: A retrospective cohort study. Lancet Respir Med. 2020; 8:1121-1131. As mentioned above, these adverse outcomes are caused by inadvertent lung injury in relation to the delivery/development of abnormal lung pressures and volume. In addition, ECMO, proning, and ICU ventilation are often limited to academic and major urban centers, where the technology and highly skilled staff are located, unlike community hospitals which are often the first port of call for many patients.

### Discussion for Acute Respiratory Distress Syndrome Caused by COVID-19

With the onset of the COVID-19 (SARS-CoV-2) pandemic in 2019, patients admitted to intensive care units (ICUs) with acute respiratory distress syndrome (ARDS) worldwide have substantially increased from previous years [1-3]. This dramatic increase in ARDS patients had overwhelmed many hospitals across the globe. Lung inflammation and fluid build-up due to ARDS can prohibit proper oxygenation and cause patients to exhibit low partial pressure of oxygen in their bloodstream, leading to hypoxemia. Roughly 25% of patients admitted to hospitals with COVID-19 develop ARDS and are transferred to ICUs [4]. The mortality rate of patients with ARDS caused by COVID-19 treated with conventional therapy may be upwards of 23% but drastically varies between studies [5, 6].

ARDS Patients admitted to hospital ICUs are typically placed on mechanical ventilators if their condition is severe enough. Even on mechanical ventilation, some patients with ARDS cannot recover from hypoxemia since the mechanical ventilator can only provide oxygen to the lungs and depends on the pulmonary system to uptake this oxygen [7]. Ventilation through the alveoli is decreased wherever there is inflammation while perfusion remains normal, leading to a decrease in the ventilation-perfusion ratio. This is referred to as a ventilation-perfusion mismatch (V/Q mismatch) and leads to lower overall blood oxygenation levels [8]. The common metric used to determine a V/Q mismatch is the P/F ratio, the partial pressure of oxygen in the arterial blood (PaO₂) normalized by the fraction of inspired oxygen (FiO₂) [9]. A P/F ratio between 200 and 300 mmHg is considered a mild case of ARDS. A P/F ratio between 100 and 200 mmHg is considered a moderate ARDS case. A severe case of ARDS is classified as a P/F ratio of less than 100 mmHg [10].

There are low-cost, conventional techniques utilized to improve the symptoms of ARDS experienced by mechanically ventilated patients with a V/Q mismatch. The first conventional method is to increase the FiO₂ delivered to the patient or to raise the mean airway pressure through higher positive and expiratory pressure. This increase in mean airway pressure has been shown to increase ventilation of inflamed alveoli or typically termed, the recruitment alveoli to participate in the gas exchange process [11]. This method does not significantly improve the P/F ratio of patients but typically increases their PaO₂. Another method is to “prone” the patient, meaning to flip the patient from a supine position to a prone position. Since the systolic pulmonary artery pressure of humans is around 20 to 25 mmHg, the potential pressure energy produced by gravity can significantly affect where perfusion is highest [12]. When someone is in the prone position, perfusion pressure is decreased in the posterior portions of the lungs and increased in the anterior portions of the lungs. Proning allows hospital staff to manipulate this pressure difference caused by gravity to increase perfusion to areas where there is increased ventilation and decrease perfusion where there is decreased ventilation. However, the proning maneuver can take up to seven hospital staff to safely carry out and does increase the risk of complications [13, 14].

More expensive, nonconventional solutions for overcoming a V/Q mismatch are the Rotoprone and ECMO machines [15, 16]. The Rotoprone machine is a rentable machine that can rotate patients to the ideal position instead of having hospital staff members perform a proning maneuver. These machines can cost about US$1,000 to rent every day and are in very limited supply [13]. Furthermore, Rotoprone machines are tedious, still requiring multiple hospital staff members to place a patient in one safely. Rotoprone machines also limit access to the patient for physical examination, blood draws, and other examinations. Extracorporeal membrane oxygenation (ECMO) systems take blood from patients, oxygenate the blood, and then return it to the patient. ECMO machines bypass the pulmonary system of the patient. The cost of an ECMO procedure was investigated in 2006 to be about US$73,000, not including the pre- and post-ECMO procedures [17].

Another more current research technique for improving the P/F ratio in patients with ARDS is to increase chest wall elastance (stiffness) [18]. This primitive technique is to simply place weights onto patients' chests while they are in the supine position [19]. The resultant effect of this method is an increase in plateau pressure (Pₚᵢₜ) of the patients while mitigating overdistension which has been shown to increase the alveolus recruitment [20]. The change in chest wall elastance from this method is only local to where the weight is placed and, therefore, is not efficient at decreasing the overall distension of patients' lungs.

With these techniques in mind, the exemplary V/Q Vest was developed to enhance the effects of the chest weight experiments by decreasing static lung compliance. The V/Q Vest has many benefits over previous methods for improving ARDS patients' condition. The V/Q Vest can be more easily applied to patients than performing a proning maneuver, takes minimal training to control and monitor, is extremely cost-effective to manufacture (about US$200), and can be manufactured on a larger scale than nonconventional treatments for a V/Q mismatch. It is hypothesized that the V/Q Vest can be used as a surrogate for other anterior chest wall compression devices and proning by adjusting the pressure the V/Q Vest applies to the chest wall of patients. Secondly, it is hypothesized that the V/Q Vest could be used to determine how a patient will respond to proning. Lastly, it is expected that the V/Q Vest will decrease the overdistension of alveoli in ARDS patients while allowing higher mean airway pressures. The V/Q Vest will be evaluated based on patient performance compared to proning these same patients.

### Additional Discussion for Acute Respiratory Distress Syndrome

A study published in 2005 estimated that around 190,000 patients are admitted to the United States intensive care units (ICUs) every year with symptoms of acute respiratory distress syndrome (ARDS) [1′-3′]. With the outbreak of the human coronavirus COVID-19 disease worldwide in 2020, the number of patients admitted to ICUs with ARDS had substantially increased. The large influx of patients admitted to ICUs in 2020 had overwhelmed many countries' healthcare systems which could be contributing to an increase in mortality rates for patients with ARDS and COVID-19.

Patients who were admitted to ICUs with severe pulmonary inflammation typically were provided oxygen via cannula and in severe presentations, patients were placed on mechanical ventilators, both of which help supply oxygen to the patient's lungs. However, in many presentations of ARDS, even the patients on mechanical ventilation failed to improve significantly due to inadequate change in ventilation-perfusion mismatch. Even though ample levels of oxygen were supplied to patients' lungs, the inflamed alveoli were unable to capture this oxygen as they were being bypassed. This phenomenon is called a ventilation-perfusion mismatch (V/Q mismatch) [4′,5′]. A V/Q mismatch potentially results when the partial pressure of oxygen in arterial blood (PaO2) decreases, and the partial pressure of carbon dioxide (PaCO2) consequently rises. Normal PaO2 levels are between 80-100 mmHg, and as levels fall to 50 mmHg hypoxemia occurs. However, PaO2 levels alone do not represent V/Q mismatch as the underlying mechanism, and hence the P/F ratio has been introduced as a descriptor of ventilation-perfusion [6′]. The P/F ratio is defined by the partial pressure of oxygen in the arterial blood divided by the fraction of inspired oxygen (FiO2).

Patients who were experiencing severe ARDS and were unresponsive to traditional mechanical ventilator maneuvers to optimize oxygenation were rotated from a supine position to a prone position. This process is called “proning.” Patients who were proned were usually sedated, chemically paralyzed, on mechanical ventilation, and provided internal fluids via intravenous therapy (IV therapy). Their care was extremely labor-intensive and complex as the patients are very tenuous. As such, great caution is required in their care, and concerning proning, up to eight hospital staff are needed to safely carry out the proning process [7′]. Risks included loss of the endotracheal tube (the tube that connects the patient to the ventilator), accidental removal of venous catheters, infection, pressure injury, etc. The potential for serious injury can be considerable [8′].

The exemplary pneumatic compression device can be used as a surrogate to proning to minimize the risk of complications while maximizing the benefits of proning. However, this device does not biomechanically replicate proning. The implications of a safer alternative to proning could be very impactful. If fewer hospital staff are needed to treat severe presentations of ARDS symptoms, hospitals could run more efficiently when dealing with an overwhelming number of patients. There also is a market gap for low-cost devices that can improve a V/Q mismatch. Currently, there are no low-cost devices on the market that are meant to improve a V/Q mismatch of patients with ARDS. As stated previously, the most common method for treating a V/Q mismatch is to prone the patient or use a very expensive and limited supply of RotoProne or ECMO systems [9′, 10′].

Although specific embodiments of the disclosure have been described, one of ordinary skill in the art will recognize that numerous other modifications and alternative embodiments are within the scope of the disclosure. For example, while various illustrative implementations and structures have been described in accordance with embodiments of the disclosure, one of ordinary skill in the art will appreciate that numerous other modifications to the illustrative implementations and structures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or another organism, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.”

It should be appreciated that, as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to humans (e.g., rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

All references cited and discussed in this specification are incorporated herein by reference in their entireties and to the same extent as if each reference was individually incorporated by reference.

### REFERENCES #1

[1] Rubenfeld, G. D., E. Caldwell, E. Peabody, J. Weaver, D. P. Martin, M. Neff, E. J. Stern, and L. D. Hudson. 2005. “Incidence and outcomes of acute lung injury.” N Engl J Med 353 (16): 1685-93. https://doi.org/10.1056/NEJMoa050333.
[2] Redant, S., J. Devriendt, I. Botta, R. Attou, D. De Bels, P. M. Honore, and C. Pierrakos. 2019. “Diagnosing Acute Respiratory Distress Syndrome with the Berlin Definition: Which Technical Investigations Should be the Best to Confirm it?” J Transl Int Med 7 (1): 1-2. https://doi.org/10.2478/jtim-2019-0001.
[3] Bourenne, J., J. Carvelli, and L. Papazian. 2019. “Evolving definition of acute respiratory distress syndrome.” J Thorac Dis 11 (Suppl 3): S390-S393. https://doi.org/10.21037/jtd.2018.12.24.
[4] Tzotzos, S. J., B. Fischer, H. Fischer, and M. Zeitlinger. 2020. “Incidence of ARDS and outcomes in hospitalized patients with COVID-19: a global literature survey.” Crit Care 24 (1): 516. https://doi.org/10.1186/s13054-020-03240-7
[5] Wunsch, H. 2020. “Mechanical Ventilation in COVID-19: Interpreting the Current Epidemiology.” Am J Respir Crit Care Med 202 (1): 1-4. https://doi.org/10.1164/rccm.202004-1385ED
[6] Hasar, S. S, T. Capstick, R. Ahmed, C. S, Kow, F. Mazhar, H. A. Merchant, & S. Zaidi. 2020 “Mortality in COVID-19 patients with acute respiratory distress syndrome and corticosteroids use: a systematic review and meta-analysis.” Expert review of respiratory medicine, 14 (11): 1149-1163. https://doi.org/10.1080/17446348.2020.1804265.
[7] Mahmood, N. A., F. A. Chaudry, H. Azam, M. I. Ali, and M. A. Khan. 2013. “Frequency of hypoxic events in patients on a mechanical ventilator.” Int J Crit Illn Inj Sci 3 (2): 124-9. https://doi.org/10.4103/2229-5151.114272.
[8] Santamarina, M. G., D. Boisier, R. Contreras, M. Baque, M. Volpacchio, and I. Beddings. 2020. “COVID-19: a hypothesis regarding the ventilation-perfusion mismatch.” Crit Care 24 (1): 395. https://doi.org/10.1186/s13054-020-03125-9.
[9] Rice, T. W., A. P. Wheeler, G. R. Bernard, D. L. Hayden, D. A. Schoenfeld, L. B. Ware, and National Heart National Institutes of Health, L.ng, and Blood Institute ARDS Network. 2007. “Comparison of the SpO2/FIO2 ratio and the PaO2/FIO2 ratio in patients with acute lung injury or ARDS.” Chest 132 (2): 410-7. https://doi.org/10.1378/chest.07-0617.
[10] Ferguson, N. D., E. Fan, L. Camporota, M. Antonelli, A. Anzueto, R. Beale, L. Brochard, R. Brower, A. Esteban, L. Gattinoni, A. Rhodes, A. S. Slutsky, J. L. Vincent, G. D. Rubenfeld, B. T. Thompson, and V. M. Ranieri. 2012. “The Berlin definition of ARDS: an expanded rationale, justification, and supplementary material.” Intensive Care Med 38 (10): 1573-82. https://doi.org/10.1007/s00134-012-2682-1.
[11] Bein, T., S. Grasso, O. Moerer, M. Quintel, C. Guerin, M. Deja, A. Brondani, & S. Mehta. 2016. “The standard of care of patients with ARDS: ventilatory settings and rescue therapies for refractory hvpoxenia.” Intensive care medicine, 42 (5): 699-711. https://doi.org/10.1007/s00134-016-4325-4.
[12] Hopkins, S. R., A. C. Henderson, D. L. Levin, K. Yamada, T. Arai, R. B. Buxton, and G. K. Prisk. 2007. “Vertical gradients in regional lung density and perfusion in the supine human lung: the Slinky effect.” J Appl Physiol (1985) 103 (1): 240-8. https://doi.org/10.1152/japplphysiol.01289.2006
[13] Wiggermann, N., J. Zhou, and D. Kumpar. 2020. “Proning Patients With COVID-19: A Review of Equipment and Methods.” Hum Factors: 18720820950532. https://doi.org/10.1177/0018720820950532.
[14] Berkow, L., and A. Kanowitz. 2020. “COVID-19 Putting Patients at Risk of Unplanned Extubation and Airway Providers at Increased Risk of Contamination.” Anesth Analg 131 (1): e41-e43. https://doi.org/10.1213/ANE.00000000004890.
[15] Baacke, M., T. Neubert, M. Spies et al. 2002. “Rotoprone®: a new and promising way to prone positioning.” Crit Care 6, P19. https://doi.org/10.1186/cc1651
[16] Shaefi, S., S. K. Brenner, S. Gupta, B. P. O'Gara, M. L. Krajewski et al. 2021. “Extracorporeal membrane oxygenation in patients with severe respiratory failure from COVID-19.” Intensive Care Med 47 (2): 208-221. https://doi.org/10.1007/s00134-020-06331-9
[17] Mishra, V., J. L. Svennevig, J. F. Bugge, S. Andresen, A. Mathisen, H. Karlsen, I. Khushi, and T. P. Hagen. 2010. “Cost of extracorporeal membrane oxygenation: evidence from the Rikshospitalet University Hospital, Oslo, Norway.” Eur J Cardiothorac Surg 37 (2): 339-42. https://doi.org/10.1016/j.ejcts.2009.06.059
[18] Gattinoni, L., D. Chiumello, E. Carlesso, and F. Valenza. 2004. “Bench-to-bedside review: chest wall elastance in acute lung injury/acute respiratory distress syndrome patients.” Crit Care 8 (5): 350-5. https://doi.org/10.1186/cc2854.
[19] Samanta, S., and K. D. Soni. 2014. “Supine chest compression: alternative to prone ventilation in acute respiratory distress syndrome.” Am J Emerg Med 32 (5): 489.e5-6. https://doi.org/10.1016/j.ajem.2013.11.014. https://www.ncbi.nlm.nih.gov/pubmed/24332252.
[20] Carteaux, G., S. Tuffet, and A. Mekontso Dessap. 2021. “Potential protective effects of continuous anterior chest compression in the acute respiratory distress syndrome: physiology of an illustrative case.” Crit Care 25 (1): 187. https://doi.org/10.1186/si3054-021-03619-0.
[21] Ambrose, A. B., L. Tiziani, D. I Ward, M. Weinmann, & F. L. Hammond III. 2021. “A Pneumatic Compression Vest for Transthoracic Manipulation of Ventilation-Perfusion in Critical Care Patients with Acute Respiratory Distress Syndrome Caused by COVID-19.” Proceedings of the 2021 Design of Medical Devices Conference. 2021 Design of MedicalDevices Conference. Minneapolis, MN, USA. Apr. 12-15, 2021. V001T04A007. ASME. https://doi.org/10.1115/DMD2021-1056
[22] Gao, M., Piernas, C., Astbury, N. M. et al. 2021. “Associations between body-mass index and COVID-19 severity in 6·9 million people in England: a prospective, community-based, cohort study.” The Lancet Diabetes & Endocrinology 9 (6):350-9. https://doi.org/10.1016/S2213-8587(21)00089-9.
[23] Vandenbunder, B., S. Ehrmann, M. Piagnerelli, B. Sauneuf, N. Serck et al. 2021. “Static compliance of the respiratory system in COVID-19 related ARDS: an international multicenter study.” Crit Care 25 (1): 52. https://doi.org/10.1186/s13054-020-03433-0.
[24] Gattinoni, L., D. Chiumello, E. Carlesso, and F. Valenza. 2004. “Bench-to-bedside review: chest wall elastance in acute lung injury/acute respiratory distress syndrome patients.” Crit Care 8 (5): 350-5. https://doi.org/10.1186/cc2854.
[25] Desai J P, Moustarah F. Pulmonary Compliance. [Updated 2020 Sep. 18]. In: StatPearls [Internet]. Treasure Island (FL): StadPearls Publishing; 2021 January. Available from: https://www.ncbi.nlm.nih.gov/books/NBK538324/
[26] Nardelli, L. M., C. S. Garcia, C. P. Pissaro, and P. R. Rocco. 2007. “Understanding the mechanisms of ventilator-induced lung injury.” Rev Bras Ter Intensiva 19 (4): 469-74. PMID: 25310165.
[27] Mion, D., and A. M. Pierin. 1998. “How accurate are sphygmomanometers?” J Hum Hypertens 12 (4): 245-8. https://doi.org/10.1038/sj.jhh.1000589.

### REFERENCES #2

[1′] Rubenfeld, G. D., Caldwell, E., Peabody, E., Weaver, J., Martin, D. P., Neff, M., Stern, E. J., & Hudson, L. D. “Incidence and outcomes of acute lung injury,” N Engl J Med Vol. 353 No. 16 (2005): pp. 1685-1693. https://doi.org/10.1056/NEJMoa050333
[2′] Redant, S., Devriendt, J., Botta, I., Attou, R., De Bels, D., Honore, P. M., & Pierrakos, C.
“Diagnosing Acute Respiratory Distress Syndrome with the Berlin Definition: Which Technical Investigations Should be the Best to Confirm it?” J Transl Int Med Vol. 7 No. 1 (2019): pp: 1-2. https://doi.org/10.2478/jtim-2019-0001
[3′] Bourenne, J., Carvelli, J., & Papazian, L. “Evolving definition of acute respiratory distress syndrome,” J Thorac Dis Vol. 11. Suppl. 3 (2019): pp. S390-S393. https://doi.org/10.21037/jtd.2018.12.24
[4′] Santamarina, M. G., Boisier, D., Contreras, R., Baque, M., Volpacchio, M., & Beddings, I. “COVID-19: a hypothesis regarding the ventilation-perfusion mismatch,” Crit Care Vol. 24 No. 1 (2020): pp. 395. https://doi.org/10.1186/s13054-020-03125-9
[5′] Barnat, N., Ghavam, S., Liu, Y et al. “Reliability of a Noninvasive Measure of V/Q Mismatch for Bronchopulmonary Dysplasia,” Ann Amer Thoracic Soc Vol. 12 No. 5 (2015): pp. 727-733. https://doi.org/10.1513/AnnalsATS.201410-462OC
[6′] Rice, T., Wheeler, A., Gordon, R. et al. “Comparison of the Spo2/Fio2 Ratio and the Pao2/Fio2 Ratio in Patients with Acute Lung Injury or ARDS,” Chest Vol. 132 No. 2 (2007): pp. 410-417. https://doi.org/10.1378/chest.07-0617
[7′] Wiggermann, N., Zhou, J., & Kumpar, D. “Proning Patients With COVID-19: A Review of Equipment and Methods,” Hum Factors Vol. 62 No. 7 (2020): pp. 1069-1076. https://doi.org/10.1177/0018720820950532
[8′] Cotton, S., Zawaydeh, Q., LeBlanc, S., Husain, A., & Malhotra, A. “Proning during covid-19: Challenges and solutions,” Heart Lung Vol. 49 No. 6 (2020): pp. 686-687. https://doi.org/10.1016/j.hrtlng.2020.08.006
[9′] Slessarev, M., Cheng, J., Ondrejicka, M., Arntfield, R., & Group, C. C. W. R. “Patient self-proning with high-flow nasal cannula improves oxygenation in COVID-19 pneumonia,” Can J Anaesth Vol 67 No. 9 (2020): pp. 1288-1290. https://doi.org/10.1007/s12630-020-01661-0
[10′] Muratore, C. S., Kharasch, V., Lund, D. P. et al. “Pulomary Morbidity in 100 Survivors of Congenital Diaphragmatic Hernia monitored in Multidisciplinary Clinic,” Journal of Ped Sur Vol. 36 No. 1 (2001): pp. 133-140. https://doi.org/10.1053/jpsu.2001.20031

### Additional Embodiments

Embodiment 1. A system for oxygenating a patient, the system comprising:
a ventilator configured for delivering varying concentrations of oxygen to the patient's lungs;
a ventilation-perfusion vest configured for applying pressure to the patient's thorax; and
a controller in operable communication with the ventilator and the ventilation-perfusion vest, the controller configured to regulate the pressure applied by the ventilation-perfusion vest to the patient's thorax.
   Embodiment 2. The system of Embodiment 1, wherein the controller is further configured to:
receive oxygenation data indicative of a detected oxygen level in the patient's body; and
regulate the pressure applied by the ventilation-perfusion vest to the patient's thorax based at least in part on the detected oxygen level.
   Embodiment 3. The system of Embodiment 2, wherein the controller is further configured to:
receive a target oxygen level for the patient; and
regulate the pressure applied by the ventilation-perfusion vest to the patient's thorax based at least in part on the detected oxygen level and the target oxygen level.
   Embodiment 4. The system of Embodiment 3, wherein the controller is further configured to:
determine that the detected oxygen level is greater than the target oxygen level; and
cause the pressure applied by the ventilation-perfusion vest to the patient's thorax to be decreased based at least in part on the determination that the detected oxygen level is greater than the target oxygen level.
   Embodiment 5. The system of Embodiment 3, wherein the controller is further configured to:
determine that the detected oxygen level is less than the target oxygen level; and
cause the pressure applied by the ventilation-perfusion vest to the patient's thorax to be increased based at least in part on the determination that the detected oxygen level is less than the target oxygen level.
   Embodiment 6. The system of any one of Embodiments 2-5, further comprising a blood oxygen monitor configured for detecting an oxygen level of the patient's blood, wherein the controller is in operable communication with the blood oxygen monitor, and wherein the controller is further configured to receive the oxygenation data from the blood oxygen monitor.
   Embodiment 7. The system of any one of Embodiments 2-5, further comprising a user interface configured for allowing a clinician to input the oxygenation data, wherein the controller is in operable communication with the user interface, and wherein the controller is further configured to receive the oxygenation data from the user interface.
   Embodiment 8. The system of any one of Embodiments 1-7, further comprising a cardiovascular pressure monitor configured for detecting a cardiovascular pressure of the patient, wherein the controller is in operable communication with the cardiovascular pressure monitor, and wherein the controller is further configured to regulate the pressure applied by the ventilation-perfusion vest to the patient's thorax based at least in part on the cardiovascular pressure.
   Embodiment 9. The system of any one of Embodiments 1-8, further comprising an esophageal pressure monitor configured for detecting an esophageal pressure of the patient, wherein the controller is in operable communication with the esophageal pressure monitor, and wherein the controller is further configured to regulate the pressure applied by the ventilation-perfusion vest to the patient's thorax based at least in part on the esophageal pressure.
   Embodiment 10. The system of any one of Embodiments 1-9, further comprising one or more pressure sensors disposed on an undersurface of the ventilation-perfusion vest and configured for detecting a localized transcutaneous pressure applied to the patient, wherein the controller is in operable communication with the one or more pressure sensors, and wherein the controller is further configured to regulate the pressure applied by the ventilation-perfusion vest to the patient's thorax based at least in part on the localized transcutaneous pressure.
   Embodiment 11. The system of any one of Embodiments 1-10, wherein the controller is further configured to:
receive ventilator data indicative of one or more operating parameters of the ventilator; and
regulate the pressure applied by the ventilation-perfusion vest to the patient's thorax based at least in part on the one or more operating parameters of the ventilator.
   Embodiment 12. The system of Embodiment 11, wherein the controller is further configured to receive the ventilator data from the ventilator.
   Embodiment 13. The system of Embodiment 11, further comprising a user interface configured for allowing a clinician to input the ventilator data, wherein the controller is in operable communication with the user interface, and wherein the controller is further configured to receive the ventilator data from the user interface.
   Embodiment 14. The system of any one of Embodiments 11-13, wherein the one or more operating parameters of the ventilator comprises one or more of a ventilator mode, a pressure, a rate, a tidal volume, a peak flow, a positive end expiratory pressure, a fractional concentration of oxygen, and an inspiratory time.
   Embodiment 15. The system of any one of Embodiments 1-14, wherein the controller is further configured to:
determine a respiratory cycle of the patient corresponding to the oxygen delivered by the ventilator to the patient's lungs; and
regulate the pressure applied by the ventilation-perfusion vest to the patient's thorax based at least in part on the respiratory cycle.
   Embodiment 16. The system of Embodiment 15, wherein the controller is further configured to decrease the pressure applied by the ventilation-perfusion vest to the patient's thorax at a beginning of the respiratory cycle.
   Embodiment 17. The system of Embodiment 15 or Embodiment 16, wherein the controller is further configured to increase the pressure applied by the ventilation-perfusion vest to the patient's thorax at an end of the respiratory cycle.
   Embodiment 18. The system of any one of Embodiments 1-17, wherein the ventilation-perfusion vest comprises one or more chambers configured for being inflated and deflated to apply pressure to the patient's thorax.
   Embodiment 19. The system of Embodiment 18, wherein the controller is further configured to regulate the pressure applied by the ventilation-perfusion vest to the patient's thorax by regulating an inflation pressure of the ventilation-perfusion vest.
   Embodiment 20. The system of Embodiment 19, wherein the controller is further configured to:
receive one or more inflation pressure settings; and
regulate the inflation pressure of the ventilation-perfusion vest based at least in part on the one or more inflation pressure settings.
   Embodiment 21. The system of Embodiment 20, wherein the one or more inflation pressure settings comprises a maximum inflation pressure setting corresponding to a maximum inflation pressure for the ventilation-perfusion vest, and wherein the controller is further configured to:
determine that the inflation pressure of the ventilation-perfusion vest is greater than the maximum inflation pressure; and
cause the inflation pressure of the ventilation-perfusion vest to be decreased below the maximum inflation pressure.
   Embodiment 22. The system of Embodiment 21, wherein the controller is further configured to cause an alarm to be activated based at least in part on the determination that the inflation pressure of the ventilation-perfusion vest is greater than the maximum inflation pressure.
   Embodiment 23. The system of any one of Embodiments 20-22, wherein the one or more inflation pressure settings comprises a minimum inflation pressure setting corresponding to a minimum inflation pressure for the ventilation-perfusion vest, and wherein the controller is further configured to:
determine that the inflation pressure of the ventilation-perfusion vest is less than the minimum inflation pressure; and
cause the inflation pressure of the ventilation-perfusion vest to be increased above the minimum inflation pressure.
   Embodiment 24. The system of Embodiment 23, wherein the controller is further configured to cause an alarm to be activated based at least in part on the determination that the inflation pressure of the ventilation-perfusion vest is less than the minimum inflation pressure.
   Embodiment 25. The system of any one of Embodiments 20-24, wherein the controller is further configured to incrementally regulate the inflation pressure of the ventilation-perfusion vest based at least in part on the one or more inflation pressure settings.
   Embodiment 26. The system of any one of Embodiments 20-25, further comprising a user interface configured for allowing a clinician to input the one or more inflation pressure settings, wherein the controller is in operable communication with the user interface, and wherein the controller is further configured to receive the one or more inflation pressure settings from the user interface.
   Embodiment 27. The system of any one of Embodiments 18-26, further comprising a pump in fluid communication with the one or more chambers, wherein the controller is in operable communication with the pump, and wherein the controller is further configured to control the pump to inflate and deflate the one or more chambers.
   Embodiment 28. The system of any one of Embodiments 18-27, wherein the one or more chambers comprises a plurality of chambers configured for being independently inflated and deflated to apply different pressures to different regions of the patient's thorax.
   Embodiment 29. The system of any one of Embodiments 1-28, wherein the controller is further configured to:
determine one or more operating parameters of the ventilator; and
adjust one or more operating parameters of the ventilation-perfusion vest based at least in part on the one or more operating parameters of the ventilator.
   Embodiment 30. The system of any one of Embodiments 1-29, wherein the controller is further configured to:
determine one or more operating parameters of the ventilation-perfusion vest; and adjust one or more operating parameters of the ventilator based at least in part on the one or more operating parameters of the ventilation-perfusion vest.

1. A method for improving oxygenation, being either a synergistic mode with proning or a replacement therapy, the method comprising:
receiving, by a controller operatively coupled to a wearable pneumatic vest, a control input from a local sensor, another device in communication with the controller indicative of a detected oxygen level in a patient's body or a user-selected target oxygen level received via manual input or by adjusting pneumatic vest settings;
determining, by the controller, a plurality of operating parameters for the wearable pneumatic vest based at least in part on the detected oxygen level or the user-selected target oxygen level; and
regulating, by the controller, an amount of pressure applied to at least a portion of the patient's back and/or upper abdomen by the wearable pneumatic vest based at least in part on the plurality of operating parameters.

2. The method of claim 1, further comprising:
receiving, by the controller, oxygenation data obtained via at least one other device, wherein the plurality of operating parameters are determined based at least in part on the oxygenation data.

3. The method of any of claim 1, wherein the wearable pneumatic vest comprises a plurality of inflatable bladders.

4. The method of claim 2, wherein the plurality of operating parameters comprises a duty cycle for each of a plurality of inflatable bladders.

5. The method of claim 4, wherein determining the plurality of operating parameters comprises:
identifying, by the controller and based at least in part on the oxygenation data, the detected oxygen level, or the user-selected target oxygen level, a target location of the patient's back and/or upper abdomen and a corresponding pressure value;
selecting, by the controller, at least one of the plurality of inflatable bladders associated with the target location; and
causing, by the controller, at least one of the plurality of inflatable bladders to inflate or deflate to reach the corresponding pressure value.

6. The method of claim 1, further comprising:
receiving, by the controller, sensor data for the patient;
adjusting, by the controller, the plurality of operating parameters based at least in part on the sensor data; and
modifying, by the controller, the amount of pressure applied to at least a portion of the patient's back and/or upper abdomen by the wearable pneumatic vest based at least in part on the adjusted operating parameters.

7. The method of claim 6, wherein the sensor data comprises at least one of an internal pressure value for each of a plurality of inflatable bladders, vibration data, audio data, or an applied amount of pressure to at least a portion of the patient's back and/or upper abdomen.

8. A pneumatic vest system comprising:
a device body comprising a plurality of independently controllable inflatable bladders;
a restriction element configured to secure the device body to at least a portion of a patient's back and/or upper abdomen and minimize pressure variations throughout the patient's breathing cycle; and
a controller operatively coupled to the plurality of independently controllable inflatable bladders, the controller being configured to regulate an amount of pressure applied to at least a portion of the patient's back and/or upper abdomen by controlling inflation and deflation of each of the plurality of independently controllable inflatable bladders.

9. The pneumatic vest system of claim 8, wherein the controller is configured to control inflation and deflation of each of the plurality of independently controllable inflatable bladders based at least in part on oxygenation data indicative of a detected oxygen level in the patient's body or a user-selected target oxygen level.

10. The pneumatic vest system of claim 8, wherein the restriction element comprises one or more removable straps attached to an anterior surface of the device body.

11. The pneumatic vest system of claim 8, further comprising:
at least one sensor array located on a posterior portion of the wearable pneumatic vest.

12. The pneumatic vest system of claim 11, wherein the at least one sensor array is configured to monitor at least one of a localized transcutaneous pressure value, cardiovascular pressure values, and an esophageal pressure value.

13. The pneumatic vest system of claim 12, wherein the at least one sensor array comprises at least one of a pressure sensor, a vibration sensor, and an audio sensor.

14. The pneumatic vest system of claim 8, wherein each of the plurality of independently controllable inflatable bladders is operatively coupled to a respective pressure sensor via a pneumatic connector, and wherein each respective pressure sensor is configured to monitor an internal pressure value for one of the plurality of independently controllable inflatable bladders.

15. The pneumatic vest system of claim 11, wherein the controller is further configured to control inflation and deflation of each of the plurality of independently controllable inflatable bladders based at least in part on sensor data obtained from the at least one sensor array.

16. The pneumatic vest system of claim 9, wherein the controller comprises a Proportional-Integral-Derivative (PID) controller that is configured to drive an internal pressure of each of the plurality of independently controllable inflatable bladders to a specified internal pressure based at least in part on the oxygenation data or the user selected target oxygen level.

17. The pneumatic vest system of claim 8, further comprising:
a graphical user interface in electronic communication with the controller that is configured to receive user inputs and facilitate monitoring of the patient.

18. The pneumatic vest system of claim 8, further comprising:
a ventilator configured for delivering varying concentrations of oxygen to the patient's lungs, the controller having an interface to communicate with the ventilator.

19. The system of claim 18, wherein the controller is in operable communication with at least one of a pulse oximeter, a blood oxygen monitor, and ventilation equipment to facilitate measurement of at least one of lung mechanics, impact on cardiovascular function, and intra-abdominal pressure.

20. The system of claim 19, wherein the controller is configured to regulate the pressure applied by the pneumatic vest based at least in part on one or more ventilator operating parameters (e.g., a ventilator mode, a pressure, a rate, a tidal volume, a peak flow, a positive end-expiratory pressure, a fractional concentration of oxygen, and an inspiratory time).
